(19) United States
(12) Patent Application Publication
(10) Pub. No.: US 2005/0020612 A1
(43) Pub. Date: Jan. 27, 2005

(54) 4-Aryliquinazolines and the use thereof as nhe-3 inhibitors

(76) Inventor: Rolf Gericke, Seeheim-Jugenheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/499,972

(22) PCT Filed: Nov. 29, 2002
(86) PCT No.: PCT/EP02/13530

(30) Foreign Application Priority Data
Dec. 24, 2001 (DE) 10163992.9

(51) Int. Cl. A61K 31/517

(52) U.S. Cl. 514/266.4; 544/292

(57) ABSTRACT

The invention relates to compounds of the formula (I) in which Ar is X-substituted phenyl or naphthyl, which is additionally substituted by R³ and R⁴, Y or X is H, NR⁶R⁷ or a saturated 5-7-membered ring having two N atoms, R¹, R², R³ and R⁴ are each, independently of one another, H, A, OA, Hal, CF₃, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF3, and salts and solvates thereof, and to the use thereof as NHE-3 inhibitors.

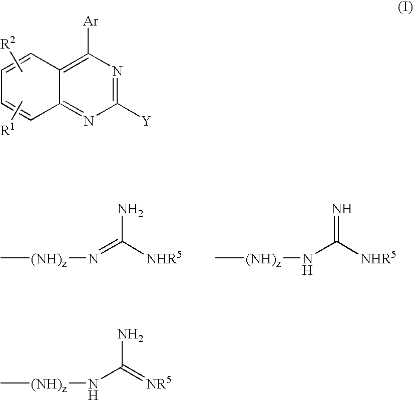

The invention relates to compounds of the formula I 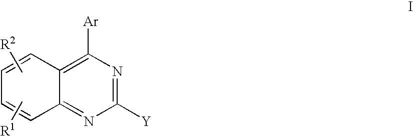
in which
Ar is X-substituted phenyl or naphthyl, which is additionally substituted by R³ and R⁴,
Y is 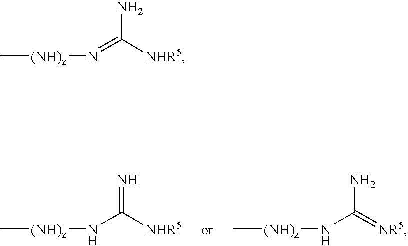
X is H, NR⁶R⁷ or a saturated 5-7-membered ring having two N atoms,
R¹, R², R³
and R⁴ are each, independently of one another, H, A, OA, Hal, CF₃, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃,
A is alkyl having 1, 2, 3, 4, 5 or 6 carbon atoms,
Hal is F, Cl, Br or I,
R⁵ is H, A, OH, NO₂, phenyl which is unsubstituted or monosubstituted or polysubstituted by A, OA, Hal or CF₃, an amino-protecting group or 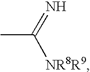
R⁶ and R⁷ are each, independently of one another, H, A, phenyl which is unsubstituted or monosubstituted or polysubstituted by A, OA, Hal or CF₃, benzyl, an amino-protecting group or —(CH₂)ₙNR¹⁰R¹¹,
R⁸ and R⁹ are each, independently of one another, H or A,
R¹⁰
and R¹¹ are each, independently of one another, H, A, phenyl which is unsubstituted or monosubstituted or polysubstituted by A, OA, Hal or CF₃, benzyl or an amino-protecting group,
Z is 0 or 1, and
n is 2, 3 or 4,
   and salts, solvates and stereoisomers thereof, including mixtures thereof in all ratios, and pharmaceutically usable derivatives thereof, in particular physiologically tolerated salts and solvates thereof,
   with the proviso that compounds in which X is H and simultaneously z is 0 are excluded.

The invention likewise relates to the use of the compounds of the formula I and their salts and solvates as NHE-3 inhibitors.

Other inhibitors of the sodium/proton exchanger subtype 3 have already been described, for example in EP 0 825 178.

The compounds excepted by the proviso have already been described in DE 10043667.

Quinazolinylguanidine derivatives have been described by V. I. Shvedov et al. in Pharm. Chem. J. (Engi. transl.) 1980, 14, 532-538 or in Khim. Farm. Zh. 1980, 14, 38-43, and by S. C. Bell et al. in J. Med. Pharm. Chem. 1962, 5, 63-69.

The invention had the object of finding novel compounds having valuable properties, in particular those which can be used for the preparation of medicaments.

Surprisingly, it has been found that the compounds of the formula I and their salts are well tolerated and inhibit sodium/proton exchanger subtype 3 and at the same time have improved bioavailability due to their increased water solubility.

The compounds of the formula I can be employed as medicament active ingredients in human and veterinary medicine.

It is known that the Na⁺/H⁺ exchanger represents a family having at least six different isoforms (NHE-1 to NHE-6), all of which have already been cloned. While subtype NHE-1 is distributed ubiquitously in all tissues throughout the body, the other NHE subtypes are expressed selectively in specific organs, such as in the kidney or in the lumen wall and contra-luminal wall of the small intestine. This distribution reflects the specific functions that the various isoforms serve, namely on the one hand regulation of the intracellular pH and cell volume by subtype NHE-1 and on the other hand Na⁺ absorption and resorption in the intestine and kidney by isoforms NHE-2 and NHE-3. Isoform NHE-4 has been found principally in the stomach. Expression of NHE-5 is restricted to the brain and neuronal tissue. NHE-6 is the isoform that forms the sodium/proton exchanger in the mitochondria.

The isoform NHE-3 is expressed in particular in the apical membrane of the proximal renal tubuli; an NHE-3 inhibitor therefore exerts, inter alia, a protective action on the kidneys.

The therapeutic use of a selective inhibitor for NHE-3 isoforms is manifold. NHE-3 inhibitors inhibit or reduce tissue damage and cell necrosis after pathophysiological hypoxic and ischaemic events which result in an activation of the NHE activity, as is the case during renal ischaemia or during the removal, transport and reperfusion of a kidney during a kidney transplant.

The compounds of the formula I have a cytoprotective action in that they prevent the excessive absorption of sodium and water into the cells of organs undersupplied with oxygen.

The compounds of the formula I have a hypotensive action and are suitable as medicament active ingredients for the treatment of hypertonia. They are furthermore suitable as diuretics.

The compounds of the formula I, alone or in combination with NHE inhibitors of other subtype specificity, have an antiischaemic action and can be used in the case of thromboses, atherosclerosis, vascular spasms, for the protection of organs, for example kidney and liver, before and during operations, and in the case of chronic or acute renal failure.

They can furthermore be used for the treatment of strokes, cerebral oedema, ischaemia of the nervous system, various forms of shock, for example allergic, cardiological, hypovolemic or bacterial shock, and for improving breathing drive in, for example, the following states: central sleep apnoea, cot death, postoperative hypoxia and other breathing disorders.

Through combination with a carboanhydrase inhibitor, breathing activity can be further improved.

The compounds of the formula I have an inhibiting effect on the proliferation of cells, for example fibroblast cell proliferation and the proliferation of the smooth muscle cells, and can therefore be used for the treatment of illnesses in which cell proliferation is a primary or secondary cause. The compounds of the formula I can be used against delayed complications of diabetes, cancer illnesses, fibrotic illnesses, endothelial dysfunction, organ hypertrophia and hyperplasia, in particular in prostate hyperplasia or prostate hypertrophia.

They are furthermore suitable as diagnostic agents for the determination and differentiation of certain forms of hypertonia, atherosclerosis, diabetes and proliferative illnesses.

Since the compounds of the formula I also have an advantageous effect on the level of serum lipoproteins, they can be employed, alone or in combination with other medicaments, for the treatment of an increased blood fat level.

The invention relates to the use of compounds of the formula I according to Claim 1 and their physiologically acceptable salts and/or solvates for the preparation of a medicament for the treatment of thrombosis, ischaemic states of the heart, of the peripheral and central nervous system and of strokes, ischaemic states of peripheral organs and extremities and for the treatment of shock states.

The invention furthermore relates to the use of compounds of the formula I according to Claim 1 and their physiologically acceptable salts and/or solvates for the preparation of a medicament for use in surgical operations and organ transplants and for the preservation and storage of transplants for surgical measures.

The invention also relates to the use of compounds of the formula I according to Claim 1 and their physiologically acceptable salts and/or solvates for the preparation of a medicament for the treatment of illnesses in which cell proliferation is a primary or secondary cause, for the treatment or prophylaxis of disorders of fat metabolism or disturbed breathing drive.

The invention furthermore relates to the use of compounds of the formula I according to Claim 1 and their physiologically acceptable salts and/or solvates for the preparation of a medicament for the treatment of renal ischaemia, ischaemic intestinal illnesses or for the prophylaxis of acute or chronic renal illnesses.

Methods for the identification of substances which inhibit sodium/proton exchanger subtype 3 are described, for example, in U.S. Pat. No. 5,871,919.

The compounds of the formula I are, in addition, suitable for the treatment of bacterial and parasitic illnesses.

For all radicals in the compounds of the formula I which occur more than once, such as, for example, A, their meanings are independent of one another.

The term hydrates and solvates of the compounds of the formula I is taken to mean, for example, the hemi-, mono- or dihydrates, and the term solvates is taken to mean, for example, alcohol addition compounds, such as, for example, with methanol or ethanol.

In the formulae above, A is alkyl, is linear or branched and has 1, 2, 3, 4, 5 or 6 carbon atoms. A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, or 1,1,2- or 1,2,2-trimethylpropyl.

OA is preferably methoxy, ethoxy, propoxy, isopropoxy or butoxy.

Hal is preferably F, Cl or Br, but also I, in particular F, Cl or Br.

Above and below, Ph is an unsubstituted phenyl radical, unless stated otherwise.

Ar is preferably phenyl which is monosubstituted by X and, for example, A, fluorine, chlorine, bromine, iodine, methoxy, ethoxy, propoxy, butoxy or CF₃. Ar is particularly preferably one of the following radicals: 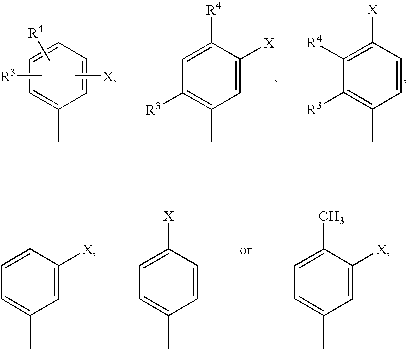
in which R³, R⁴ and X are as defined above.

X is preferably NR⁶R⁷, a 5- to 7-membered ring having 2 N atoms or the following radical: 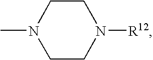
in which R¹² is H, A, Ph, benzyl or an amino-protecting group, such as, for example, BOC or CBO and in particular H, A or phenyl.

In particular, X is H, NA₂ or a radical from the following group:

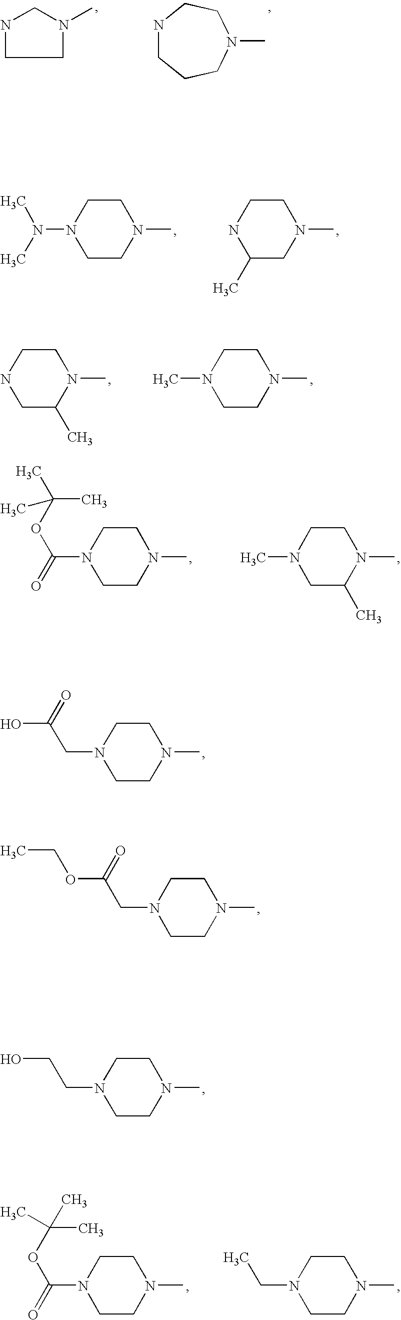

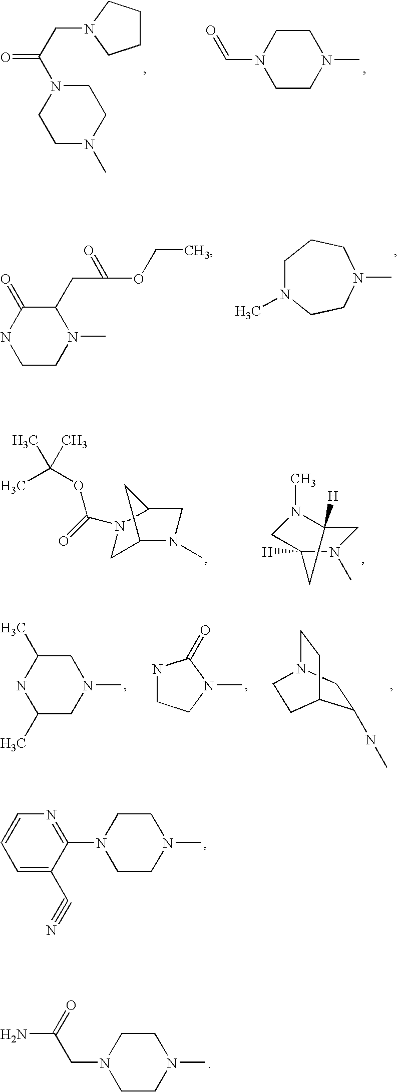

R⁵ is preferably H, A, OH, NO₂ or an amino-protecting group, in particular H, A, OH or NO₂.

R⁶ and R⁷ are preferably simultaneously H, independently of one another H, A, benzyl or —(CH₂)ₙNA₂.

R⁸ and R⁹ are preferably H or methyl, in particular H.

R¹⁰ and R¹¹ are preferably H, A, benzyl or phenyl, in particular H, methyl or benzyl.

z is preferably 0. n is preferably 2.

The term “amino-protecting group” is known in general terms and relates to groups which are suitable for protecting (blocking) an amino group against chemical reactions, but can easily be removed after the desired chemical reaction has been carried out elsewhere in the molecule. Typical of such groups are, in particular, unsubstituted or substituted acyl, aryl, aralkoxymethyl and aralkyl groups. Since the amino-protecting groups are removed after the desired reaction (or reaction sequence), their nature and size are furthermore not crucial; however, preference is given to those having 1-20, in particular 1-8, carbon atoms. The term “acyl group” covers acyl groups derived from aliphatic, araliphatic, aromatic or heterocyclic carboxylic acids or sulfonic acids and in particular alkoxycarbonyl, aryloxycarbonyl and especially aralkoxycarbonyl groups. Examples of amino-protecting groups of this type are alkanoyl, such as acetyl, propionyl and butyryl; aralkanoyl, such as phenylacetyl; aroyl, such as benzoyl or toluyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; alkenyloxycarbonyl, such as allyloxycarbonyl (Aloc), aralkyloxycarbonyl, such as CBZ (“carbobenzoxy”, synonymous with Z), 4-methoxybenzyloxycarbonyl (MOZ), 4-nitrobenzyloxycarbonyl or 9-fluorenylmethoxycarbonyl (FMOC); 2-(phenylsulfonyl)ethoxycarbonyl; trimethylsilylethoxycarbonyl (Teoc), or arylsulfonyl, such as 4-methoxy-2,3,6-trimethylphenylsulfonyl (Mtr). The amino-protecting group is preferably formyl, acetyl, propionyl, butyryl, phenylacetyl, benzoyl, toluyl, POA, methoxycarbonyl, ethoxycarbonyl, 2,2,2-trichloroethoxycarbonyl, BOC, 2-iodoethoxycarbonyl, CBZ (“carbobenzoxy”), 4-methoxybenzyloxycarbonyl, FMOC, Mtr or benzyl.

The invention relates in particular to the compounds of the formula I in which at least one of the said radicals has one of the preferred meanings indicated above, and to the use thereof.

Preference is furthermore given to compounds of the formula I and salts and solvates thereof in which at least one of the radicals R¹, R², R³ and R⁴ has one of the following meanings:

Hal, A, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃.

Of the compounds of the formula 1, particular preference is given to those whose radical R¹ is Cl, in particular in position 6, and those whose radical R³ is methyl, in particular in position 4′.

Compounds of the formula I whose radical R³ is methyl, in particular in position 4′, have particularly pronounced selectivity of binding to the NHE-3 receptor.

Preference is furthermore given to the compounds of the formulae IA, IB and IC: 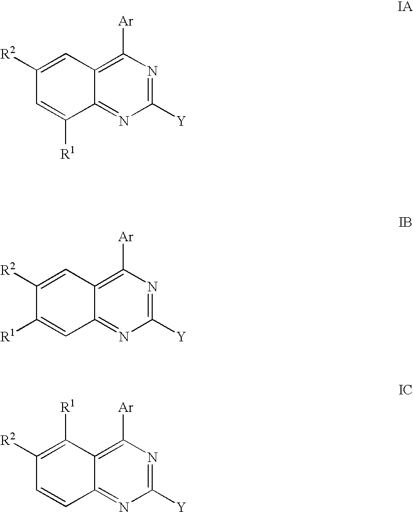
in which R¹, R², Ar and Y are as defined above.

In particular, R¹ in the formulae IA, IB and IC is H, while R² is Hal or in particular Cl.

Y preferably adopts one of the following meanings:

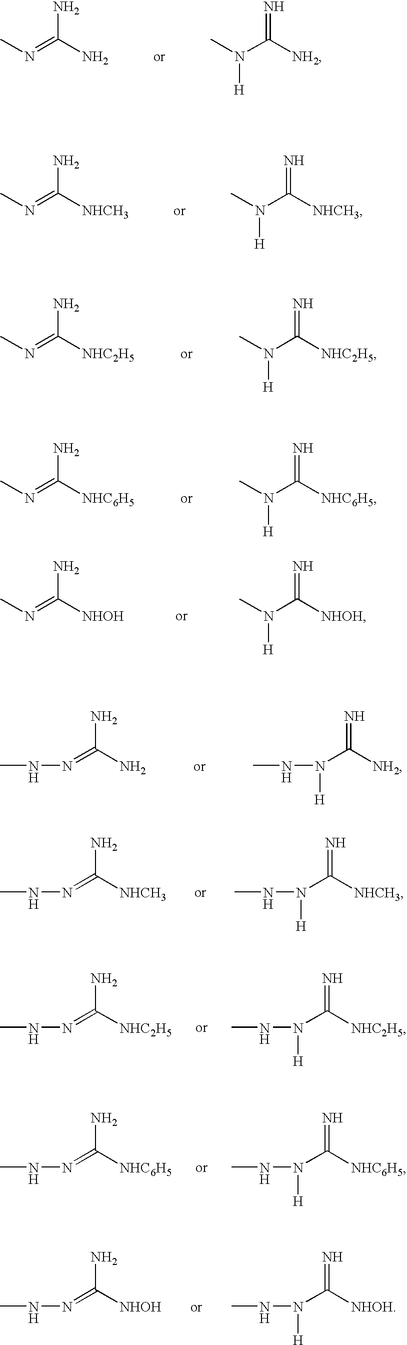

Y particularly preferably has one of the following meanings:

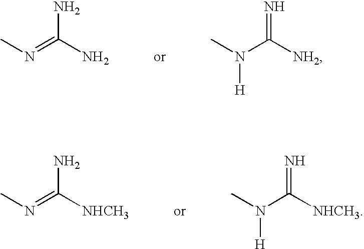

Particular preference is furthermore given to the following compounds I1 to I10 and salts and solvates thereof:

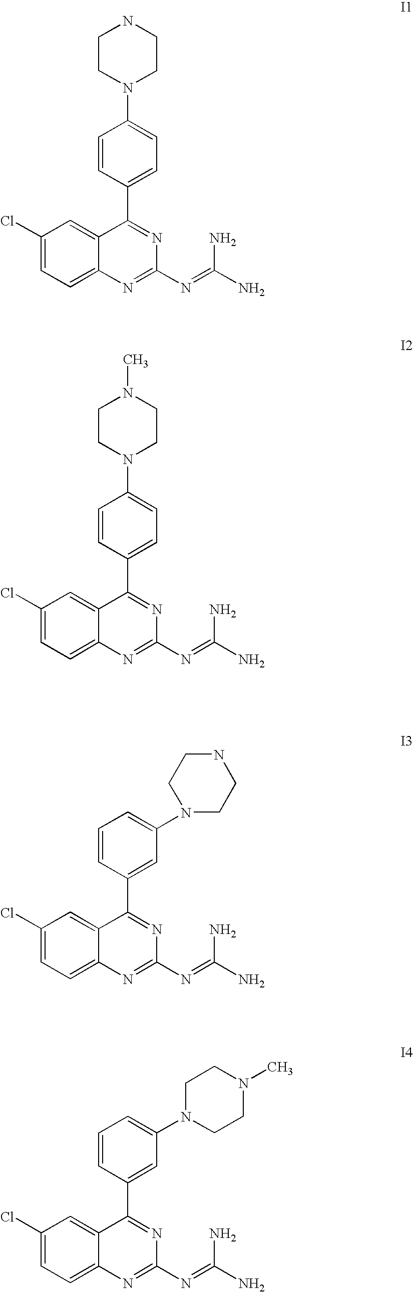

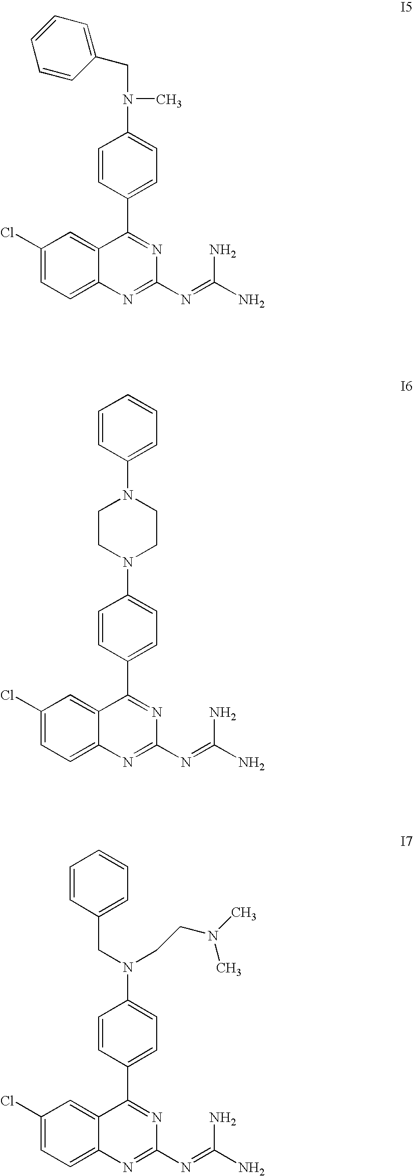

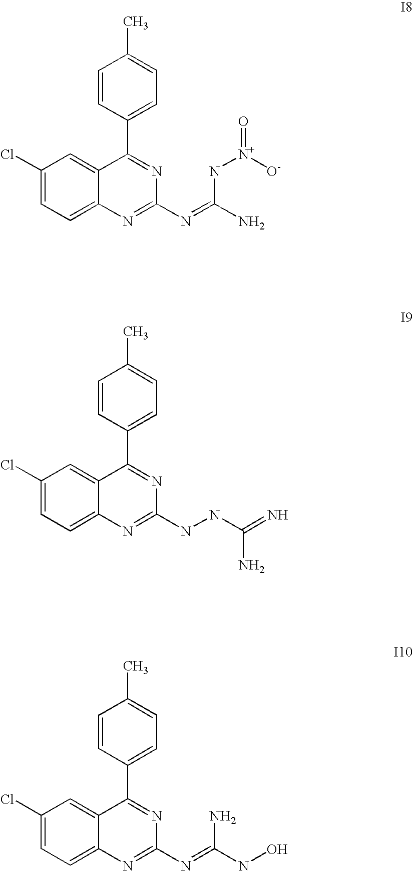

The hydrochlorides and p-toluenesulfonates of the compounds of the formulae I1 to I10 are very particularly preferred.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

The starting materials can, if desired, also be formed in situ, so that they are not isolated from the reaction mixture, but instead are immediately converted further into the compounds of the formula I.

The compounds of the formula I are preferably prepared by reacting compounds of the formula II 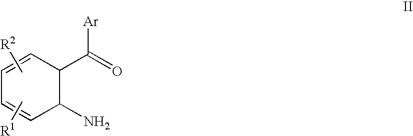
in which R¹, R² and Ar are as defined above,
with 1-cyanoguanidine or a correspondingly N-alkylated or N-arylated 1-cycanoguanidine of the formula NC—Y, in which Y is as defined above and z is 0.

The reaction can be carried out in a solvent, preferably an inert solvent.

Examples of suitable solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

DMF, water or an alcohol is preferably used.

The reaction is very particularly preferably carried out without a solvent, i.e. in the melt, at temperatures between 100 and 200° C.

The presence of an acidic catalyst, such as AlCl₃, TiCl₄, p-toluenesulfonic acid, BF₃, acetic acid, sulfuric acid, oxalic acid, POCl₃ or phosphorus pentoxide, is advantageous.

A preferred variant comprises employing at least one of the reactants already as a salt, for example as the hydrochloride.

A further valuable method for the preparation of the compounds of the formula I comprises reacting, instead of a compound of the formula NC—Y, a compound of the formula III
HN═CX—Y III
in which
X is —S-alkyl, —S-aryl, —O-alkyl or —O-aryl,
Y is as defined above, where z is 0, alkyl is preferably as defgined above for A, and aryl is as defined above for Ar,
   with a compound of the formula II.

Finally, the compounds of the formula I can be prepared by reaction of compounds of the formula IV 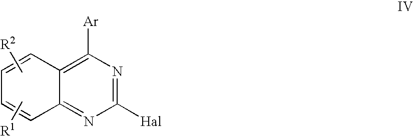
in which Ar, Hal, R¹ and R² are as defined above and Hal is in particular Cl, with a compound of the formula HY, in which Y is as defined above. HY is particularly preferably guanidine or a compound of the following formula: 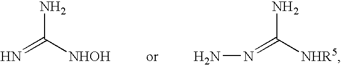
in which R⁵ is as defined above.

This reaction is preferably carried out in the presence of a strong base, such as alkali metal alkoxide or strongly basic amines. The bases used are particularly preferably sodium methoxide, sodium ethoxide, potassium methoxide, potassium ethoxide, potassium tert-butoxide, DBN, DBU or DABCO.

The solvents used for the reaction of compounds of the formula IV with compounds of the formula HY are preferably DMSO, NMP or DMF.

The compounds of the formula IV can be obtained by preparation methods which are known per se.

The compounds of the formula IV are particularly preferably prepared by reaction of the compounds of the formula V 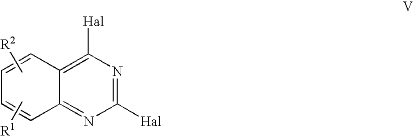
V
in which R¹, R² and Hal are as defined above,
a)
with boronic acids of the formula Ar—B(OH)₂ in the presence of a palladium compound, such as, for example, bis(triphenylphosphine)palladium(II) chloride in the form of a Suzuki coupling. Many variants of this reaction have already been disclosed in the literature (for example S. L. Buchwald and J. M. Fox, The Strem Chemiker, 200, 18, 1), Okabe et al., Tetrahedron, 1995, 51, 1861 to 1866; Curd et al. J. Chem. Soc. 1948, 1759 to 1766). or
b)
with tributyltin compounds of the formula Ar—Sn(n-C₂H₅)₃ in the form of a Stile coupling (for example J. K. Stille Angew. Chem. Int. Ed. Engl. 1986, 25, 508).

The present application likewise relates to the process for the preparation of the compounds of the formula V.

In some cases, it may be appropriate only to form the radicals R¹, R², R³ and R⁴ and other functional groups after the reaction of the compounds of the formula II with the compounds of the formula NC—Y or the compounds of the formula III, for example by removal of a protecting group, ether cleavage or hydrogenation of nitro groups to amino groups.

Correspondingly, it may likewise be appropriate only to form the radicals R¹, R², R³ and R⁴ and other functional groups after the reaction of the compounds of the formula IV with the compounds of the formula HY by the above-mentioned measures.

The compounds of the formula I in which X is NR⁶R⁷ or a saturated 5-7-membered ring having two N atoms are preferably synthesised from the halogen compounds of the formula VI 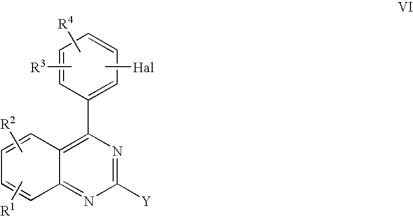
by palladium-catalysed amidation using the corresponding nitrogen bases, preferably HNR⁶R⁷ or the following nitrogen base: 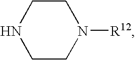
in which R⁶, R⁷ and R¹² are as defined above.

Reactions of this type have been described, for example, by Buchwald and Hartwig (R. Stürmer, Ang. Chem. 1999, 111, 3509 to 3510; L. Buchwald et al. J. Am. Chem. Soc. 1998, 120, 9722 to 9723). Besides a suitable palladium catalyst, such as, for example, Pd(OAc)₂ or Pd₂(dba)₃, the choice of ligand, in particular, is of crucial importance for the success of the reaction. Examples of suitable ligands are 2-(di-tert-butylphosphinyl)-biphenyl, 2-dimethylamino-2′-(di-tert-butylphosphinyl)biphenyl, FcPtPtBu2 or 2,2′-bis(dicyclohexylphosphino)-1,1′-binaphthyl.

Before the reaction, any free amino groups present should be protected, for example by means of amino-protecting groups.

The present application likewise relates to the novel compounds of the formulae II, IV and VI.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

The invention furthermore relates to the use of the compounds of the formula I as NHE-3 inhibitors and/or their physiologically acceptable salts for the preparation of pharmaceutical preparations, in particular by non-chemical methods. In this case, they can be converted into a suitable dosage form together with at least one solid, liquid and/or semiliquid excipient or assistant, and, if desired, in combination with one or more further active ingredients.

The invention furthermore relates to pharmaceutical preparations comprising at least one NHE-3 inhibitor of the formula I and/or one of its physiologically acceptable salts and solvates.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders, or transdermally in patches.

The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins.

Suitable pharmaceutical preparations for administration in the form of aerosols or sprays are, for example, solutions, suspensions or emulsions of the active ingredient of the formula I in a pharmaceutically acceptable solvent.

The compounds of the formula I and their physiologically acceptable salts and solvates can be used for the treatment and/or prophylaxis of the illnesses or illness states described above.

In general, the substances according to the invention are preferably administered in doses between about 0.1 and 500 mg, in particular between 1 and 10 mg, per dosage unit. The daily dose is preferably between about 0.001 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

### EXAMPLES

Above and below, all temperatures are indicated in ° C. In the following examples, “conventional work-up” means that water is added if necessary, the mixture is adjusted, if necessary, to a pH between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. m.p. stands for melting point.

### Example 1

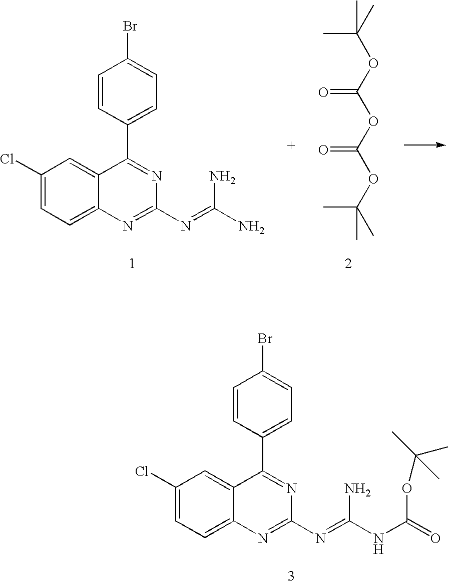

A mixture of 1.00 g of compound 1, 0.60 g of di-tert-butyl dicarbonate (2), 20.0 ml of dimethylformamide and 1.20 g of potassium carbonate is heated at 60° C. overnight. Water is added to the reaction mixture, the mixture is filtered, and the residue is subjected to conventional work-up, giving compound 3.

### Example 2

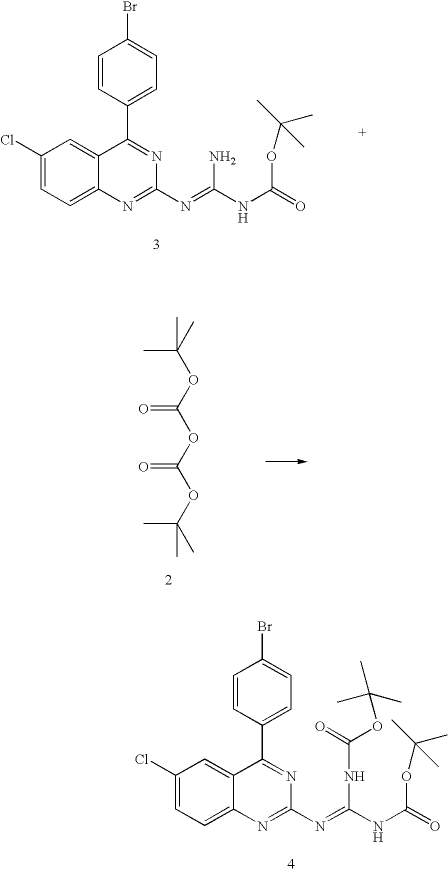

A mixture of 100 mg of compound 3, 600 mg of di-tert-butyl dicarbonate (2) and 5 ml of dichloromethane is stirred at 50° C. for 4 hours. The solvent is removed, and the residue is subjected to conventional work-up, giving compound 4.

### Example 3

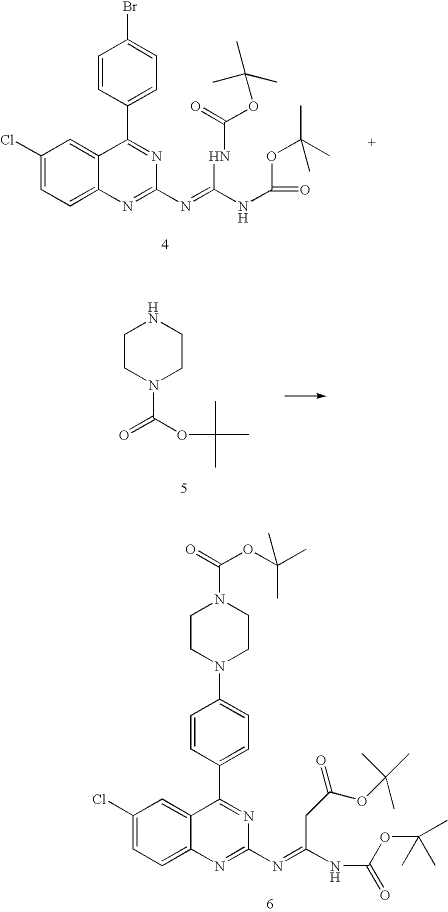

0.242 g of compound 4, 0.16 g of tert-butyl 1-piperazylcarboxylate and 0.170 g of sodium tert-butoxide are added under a nitrogen atmosphere to a solution of 60 mg of palladium(II) acetate and 100 mg of tri-tert-butylphosphine in 5 ml of xylene. The mixture is stirred at room temperature for 24 hours, water is added, and the mixture is subjected to conventional work-up, giving compound 6.

### Example 4

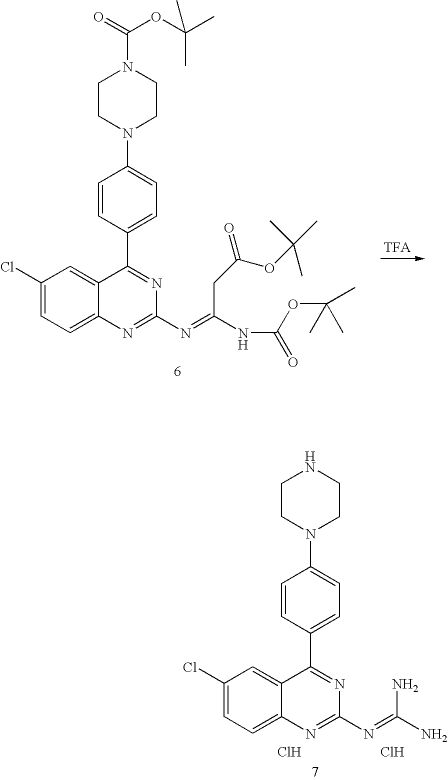

20 mg of trifluoroacetic acid are added to 1.50 g of compound 6 with ice cooling, and the mixture is stirred at room temperature for one hour. All the volatile components are removed, the residue is dissolved in 50 ml of water, and 1 N sodium hydroxide solution is added dropwise until the mixture is alkaline. The resultant precipitate is filtered off with suction, dissolved in 20 ml of methanol and precipitated by addition of methanolic hydrochloric acid solution. Filtration and conventional work-up give compound 7 (m.p. 245° C.).

### Example 5

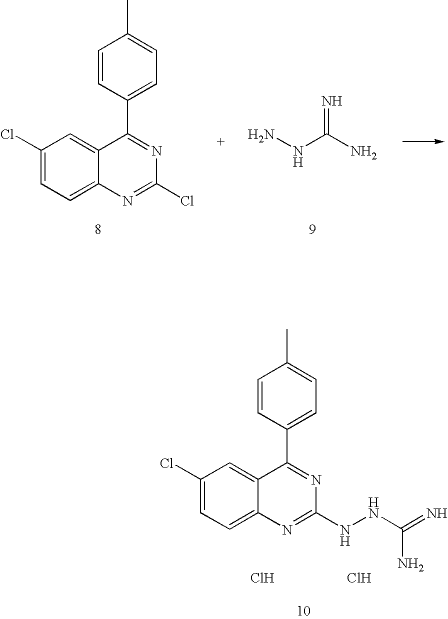

A mixture of 0.10 g of compound 8 and 0.152 g of compound 9 in 1.0 ml of 1-methyl-2-pyrrolidone is stirred at room temperature for 2 hours. The reaction mixture is subsequently filtered, the residue is dissolved in a mixture of methanol and dichloromethane, and methanolic hydrochloric acid solution is added. Cooling, re-filtration and conventional work-up give compound 10.

### Example 6

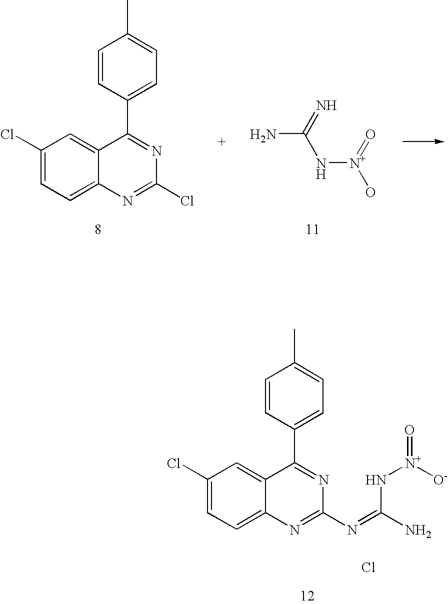

A mixture of 0.75 g of compound 8, 0.40 g of nitroguanidine (11) and 0.40 ml of DABCO in 5 ml of 1-methyl-2-pyrrolidone is stirred at 80° C. for 6 hours. Conventional work-up of the reaction mixture gives compound 12.

### Example 7

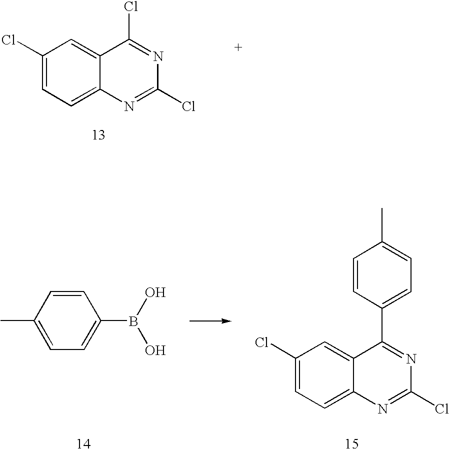

A mixture of 5.00 g of compound 13, 2.91 g of tolylboronic acid (14), 0.30 g of bis(triphenylphosphine)palladium(II) chloride and 1.2 g of powdered sodium hydroxide in 60 ml of diglyme is stirred at 130° C. for six hours. Water is subsequently added to the reaction mixture, which is subjected to conventional work-up, giving compound 15.

### Example 8

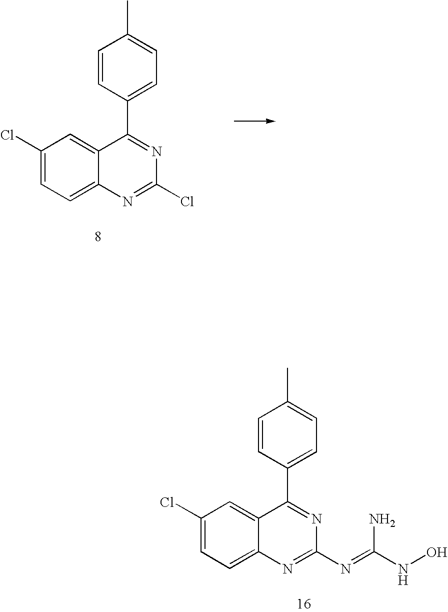

A mixture of 1.45 g of compound 8 and 0.58 g of DABCO in 15 ml of dimethyl sulfoxide is stirred at room temperature for 2 hours, and 2.66 g of hydroxyguanidine sulfate 1-hydrate are subsequently added. After 1.39 ml of triethylamine have been added dropwise, the reaction mixture is stirred at room temperature for two days. Conventional work-up of the mixture gives compound 16 (m.p. 193-195° C.).

The following preferred NHE-3 inhibitors were obtained as acid-addition salts or free bases analogously to the processes indicated above using the corresponding precursors:

pTsOH below denotes p-toluenesulfonic acid.

### Examples 9-44

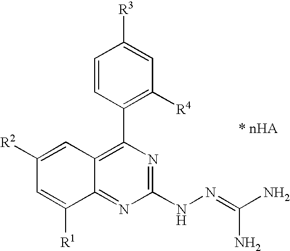

| | R¹ | R² | R³ | R⁴ | nHA | |
|---|---|---|---|---|---|---|
| (9) | H | Cl | H | H | pTsOH | |
| (10) | H | Cl | CH₃ | H | HCl | m.p. 289-290° C. |
| (11) | H | Cl | C₂H₅ | H | HCl | |
| (12) | H | Cl | OCH₃ | H | HCl | |
| (13) | H | Cl | NO₂ | H | HCl | |
| (14) | H | Cl | NH₂ | H | pTsOH | |
| (15) | H | Cl | N(CH₃)₂ | H | pTsOH | |
| (16) | H | Cl | H | NH₂ | HCl | |
| (17) | H | Cl | CH₃ | NH₂ | pTsOH | |
| (18) | H | Cl | C₂H₅ | NH₂ | HCl | |
| (19) | H | Cl | OCH₃ | NH₂ | HCl | |
| (20) | H | Cl | NO₂ | NH₂ | HCl | |
| (21) | H | Cl | NH₂ | NH₂ | HCl | |
| (22) | H | Cl | N(CH₃)₂ | NH₂ | HCl | |
| (23) | H | Cl | H | NHCH₃ | HCl | |
| (24) | H | Cl | CH₃ | NHCH₃ | HCl | |
| (25) | H | Cl | C₂H₅ | NHCH₃ | HCl | |
| (26) | H | Cl | OCH₃ | NHCH₃ | HCl | |
| (27) | H | Cl | NO₂ | NHCH₃ | HCl | |
| (28) | H | Cl | NH₂ | NHCH₃ | HCl | |
| (29) | H | Cl | N(CH₃)₂ | NHCH₃ | HCl | |
| (30) | H | Cl | H | N(CH₃)₂ | HCl | |
| (31) | H | Cl | CH₃ | N(CH₃)₂ | HCl | |
| (32) | H | Cl | C₂H₅ | N(CH₃)₂ | HCl | |
| (33) | H | Cl | OCH₃ | N(CH₃)₂ | HCl | |
| (34) | H | Cl | NO₂ | N(CH₃)₂ | HCl | |
| (35) | H | Cl | NH₂ | N(CH₃)₂ | HCl | |
| (36) | H | Cl | N(CH₃)₂ | N(CH₃)₂ | HCl | |
| (37) | H | Cl | H | OH | HCl | |
| (38) | H | Cl | CH₃ | OH | HCl | |
| (39) | H | Cl | C₂H₅ | OH | HCl | |
| (40) | H | Cl | OCH₃ | OH | HCl | |
| (41) | H | Cl | NO₂ | OH | HCl | |
| (42) | H | Cl | NH₂ | OH | HCl | |
| (43) | H | Cl | N(CH₃)₂ | OH | HCl | |
| (44) | H | Cl | SO₂CH₃ | CH₃ | HCl | |

### Examples 45-80

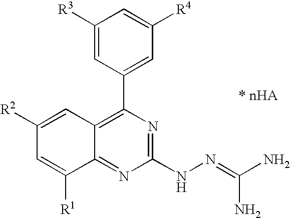

| | R¹ | R² | R³ | R⁴ | nHA |
|---|---|---|---|---|---|
| (45) | H | Cl | H | H | pTsOH |
| (46) | H | Cl | CH₃ | H | HCl |
| (47) | H | Cl | C₂H₅ | H | HCl |
| (48) | H | Cl | OCH₃ | H | HCl |
| (49) | H | Cl | NO₂ | H | HCl |
| (50) | H | Cl | NH₂ | H | pTsCO |
| (51) | H | Cl | N(CH₃)₂ | H | pTsOH |
| (52) | H | Cl | H | NH₂ | HCl |
| (53) | H | Cl | CH₃ | NH₂ | pTsOH |
| (54) | H | Cl | C₂H₅ | NH₂ | HCl |
| (55) | H | Cl | OCH₃ | NH₂ | HCl |
| (56) | H | Cl | NO₂ | NH₂ | HCl |
| (57) | H | Cl | NH₂ | NH₂ | HCl |
| (58) | H | Cl | N(CH₃)₂ | NH₂ | HCl |
| (59) | H | Cl | H | NHCH₃ | HCl |
| (60) | H | Cl | CH₃ | NHCH₃ | HCl |
| (61) | H | Cl | C₂H₅ | NHCH₃ | HCl |
| (62) | H | Cl | OCH₃ | NHCH₃ | HCl |
| (63) | H | Cl | NO₂ | NHCH₃ | HCl |
| (64) | H | Cl | NH₂ | NHCH₃ | HCl |
| (65) | H | Cl | N(CH₃)₂ | NHCH₃ | HCl |
| (66) | H | Cl | H | N(CH₃)₂ | HCl |
| (67) | H | Cl | CH₃ | N(CH₃)₂ | HCl |
| (68) | H | Cl | C₂H₅ | N(CH₃)₂ | HCl |
| (69) | H | Cl | OCH₃ | N(CH₃)₂ | HCl |
| (70) | H | Cl | NO₂ | N(CH₃)₂ | HCl |
| (71) | H | Cl | NH₂ | N(CH₃)₂ | HCl |
| (72) | H | Cl | N(CH₃)₂ | N(CH₃)₂ | HCl |
| (73) | H | Cl | H | OH | HCl |
| (74) | H | Cl | CH₃ | OH | HCl |
| (75) | H | Cl | C₂H₅ | OH | HCl |
| (76) | H | Cl | OCH₃ | OH | HCl |
| (77) | H | Cl | NO₂ | OH | HCl |
| (78) | H | Cl | NH₂ | OH | HCl |
| (79) | H | Cl | N(CH₃)₂ | OH | HCl |
| (80) | H | Cl | SO₂CH₃ | CH₃ | HCl |

### Examples 81-116

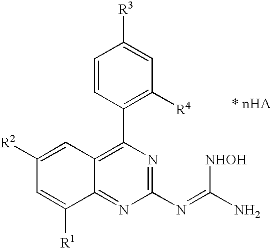

| | R¹ | R² | R³ | R⁴ | nHA |
|---|---|---|---|---|---|
| (81) | H | Cl | H | H | pTsOH |
| (82) | H | Cl | CH₃ | H | HCl |
| (83) | H | Cl | C₂H₅ | H | HCl |
| (84) | H | Cl | OCH₃ | H | HCl |
| (85) | H | Cl | NO₂ | H | HCl |
| (86) | H | Cl | NH₂ | H | pTsOH |
| (87) | H | Cl | N(CH₃)₂ | H | pTsOH |
| (88) | H | Cl | H | NH₂ | HCl |
| (89) | H | Cl | CH₃ | NH₂ | pTsOH |
| (90) | H | Cl | C₂H₅ | NH₂ | HCl |
| (91) | H | Cl | OCH₃ | NH₂ | HCl |
| (92) | H | Cl | NO₂ | NH₂ | HCl |
| (93) | H | Cl | NH₂ | NH₂ | HCl |
| (94) | H | Cl | N(CH₃)₂ | NH₂ | HCl |
| (95) | H | Cl | H | NHCH₃ | HCl |
| (96) | H | Cl | CH₃ | NHCH₃ | HCl |
| (97) | H | Cl | C₂H₅ | NHCH₃ | HCl |
| (98) | H | Cl | OCH₃ | NHCH₃ | HCl |
| (99) | H | Cl | NO₂ | NHCH₃ | HCl |
| (100) | H | Cl | NH₂ | NHCH₃ | HCl |
| (101) | H | Cl | N(CH₃)₂ | NHCH₃ | HCl |
| (102) | H | Cl | H | N(CH₃)₂ | HCl |
| (103) | H | Cl | CH₃ | N(CH₃)₂ | HCl |
| (104) | H | Cl | C₂H₅ | N(CH₃)₂ | HCl |
| (105) | H | Cl | OCH₃ | N(CH₃)₂ | HCl |
| (106) | H | Cl | NO₂ | N(CH₃)₂ | HCl |
| (107) | H | Cl | NH₂ | N(CH₃)₂ | HCl |
| (108) | H | Cl | N(CH₃)₂ | N(CH₃)₂ | HCl |
| (109) | H | Cl | H | OH | HCl |
| (110) | H | Cl | CH₃ | OH | HCl |
| (111) | H | Cl | C₂H₅ | OH | HCl |
| (112) | H | Cl | OCH₃ | OH | HCl |
| (113) | H | Cl | NO₂ | OH | HCl |
| (114) | H | Cl | NH₂ | OH | HCl |
| (115) | H | Cl | N(CH₃)₂ | OH | HCl |
| (116) | H | Cl | SO₂CH₃ | CH₃ | HCl |

### Examples 117-152

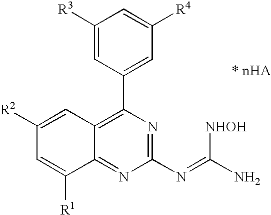

| | R¹ | R² | R³ | R⁴ | nHA |
|---|---|---|---|---|---|
| (117) | H | Cl | H | H | pTsOH |
| (118) | H | Cl | CH₃ | H | HCl |
| (119) | H | Cl | C₂H₅ | H | HCl |
| (120) | H | Cl | OCH₃ | H | HCl |
| (121) | H | Cl | NO₂ | H | HCl |
| (122) | H | Cl | NH₂ | H | pTsOH |
| (123) | H | Cl | N(CH₃)₂ | H | pTsOH |
| (124) | H | Cl | H | NH₂ | HCl |
| (125) | H | Cl | CH₃ | NH₂ | pTsOH |
| (126) | H | Cl | C₂H₅ | NH₂ | HCl |
| (127) | H | Cl | OCH₃ | NH₂ | HCl |
| (128) | H | Cl | NO₂ | NH₂ | HCl |
| (129) | H | Cl | NH₂ | NH₂ | HCl |
| (130) | H | Cl | N(CH₃)₂ | NH₂ | HCl |
| (131) | H | Cl | H | NHCH₃ | HCl |
| (132) | H | Cl | CH₃ | NHCH₃ | HCl |
| (133) | H | Cl | C₂H₅ | NHCH₃ | HCl |
| (134) | H | Cl | OCH₃ | NHCH₃ | HCl |
| (135) | H | Cl | NO₂ | NHCH₃ | HCl |
| (136) | H | Cl | NH₂ | NHCH₃ | HCl |
| (137) | H | Cl | N(CH₃)₂ | NHCH₃ | HCl |
| (138) | H | Cl | H | N(CH₃)₂ | HCl |
| (139) | H | Cl | CH₃ | N(CH₃)₂ | HCl |
| (140) | H | Cl | C₂H₅ | N(CH₃)₂ | HCl |
| (141) | H | Cl | OCH₃ | N(CH₃)₂ | HCl |
| (142) | H | Cl | NO₂ | N(CH₃)₂ | HCl |
| (143) | H | Cl | NH₂ | N(CH₃)₂ | HCl |
| (144) | H | Cl | N(CH₃)₂ | N(CH₃)₂ | HCl |
| (145) | H | Cl | H | OH | HCl |
| (146) | H | Cl | CH₃ | OH | HCl |
| (147) | H | Cl | C₂H₅ | OH | HCl |
| (148) | H | Cl | OCH₃ | OH | HCl |
| (149) | H | Cl | NO₂ | OH | HCl |
| (150) | H | Cl | NH₂ | OH | HCl |
| (151) | H | Cl | N(CH₃)₂ | OH | HCl |
| (152) | H | Cl | SO₂CH₃ | CH₃ | HCl |

### Examples 153-188

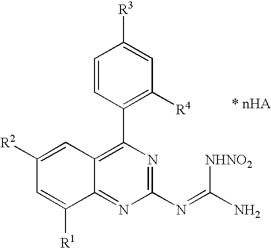

| | R¹ | R² | R³ | R⁴ | nHA |
|---|---|---|---|---|---|
| (153) | H | Cl | H | H | pTsOH |
| (154) | H | Cl | CH₃ | H | pTsOH |
| (155) | H | Cl | C₂H₅ | H | HCl |
| (156) | H | Cl | OCH₃ | H | HCl |
| (157) | H | Cl | NO₂ | H | HCl |
| (158) | H | Cl | NH₂ | H | pTsOH |
| (159) | H | Cl | N(CH₃)₂ | H | pTsOH |
| (160) | H | Cl | H | NH₂ | HCl |
| (161) | H | Cl | CH₃ | NH₂ | pTsOH |
| (162) | H | Cl | C₂H₅ | NH₂ | HCl |
| (163) | H | Cl | OCH₃ | NH₂ | HCl |
| (164) | H | Cl | NO₂ | NH₂ | HCl |
| (165) | H | Cl | NH₂ | NH₂ | HCl |
| (166) | H | Cl | N(CH₃)₂ | NH₂ | HCl |
| (167) | H | Cl | H | NHCH₃ | HCl |
| (168) | H | Cl | CH₃ | NHCH₃ | HCl |
| (169) | H | Cl | C₂H₅ | NHCH₃ | HCl |
| (170) | H | Cl | OCH₃ | NHCH₃ | HCl |
| (171) | H | Cl | NO₂ | NHCH₃ | HCl |
| (172) | H | Cl | NH₂ | NHCH₃ | HCl |
| (173) | H | Cl | N(CH₃)₂ | NHCH₃ | HCl |
| (174) | H | Cl | H | N(CH₃)₂ | HCl |
| (175) | H | Cl | CH₃ | N(CH₃)₂ | HCl |
| (176) | H | Cl | C₂H₅ | N(CH₃)₂ | HCl |
| (177) | H | Cl | OCH₃ | N(CH₃)₂ | HCl |
| (178) | H | Cl | NO₂ | N(CH₃)₂ | HCl |
| (179) | H | Cl | NH₂ | N(CH₃)₂ | HCl |
| (180) | H | Cl | N(CH₃)₂ | N(CH₃)₂ | HCl |
| (181) | H | Cl | H | OH | HCl |
| (182) | H | Cl | CH₃ | OH | HCl |
| (183) | H | Cl | C₂H₅ | OH | HCl |
| (184) | H | Cl | OCH₃ | OH | HCl |
| (185) | H | Cl | NO₂ | OH | HCl |
| (186) | H | Cl | NH₂ | OH | HCl |
| (187) | H | Cl | N(CH₃)₂ | OH | HCl |
| (188) | H | Cl | SO₂CH₃ | CH₃ | HCl |

### Examples 189-224

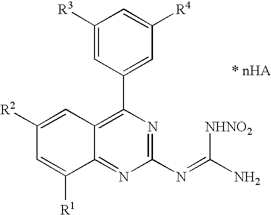

| | R¹ | R² | R³ | R⁴ | nHA |
|---|---|---|---|---|---|
| (189) | H | Cl | H | H | pTsOH |
| (190) | H | Cl | CH₃ | H | HCl |
| (191) | H | Cl | C₂H₅ | H | HCl |
| (192) | H | Cl | OCH₃ | H | HCl |
| (193) | H | Cl | NO₂ | H | HCl |
| (194) | H | Cl | NH₂ | H | pTsOH |
| (195) | H | Cl | N(CH₃)₂ | H | pTsOH |
| (196) | H | Cl | H | NH₂ | HCl |
| (197) | H | Cl | CH₃ | NH₂ | pTsOH |
| (198) | H | Cl | C₂H₅ | NH₂ | HCl |
| (199) | H | Cl | OCH₃ | NH₂ | HCl |
| (200) | H | Cl | NO₂ | NH₂ | HCl |
| (201) | H | Cl | NH₂ | NH₂ | HCl |
| (202) | H | Cl | N(CH₃)₂ | NH₂ | HCl |
| (203) | H | Cl | H | NHCH₃ | HCl |
| (204) | H | Cl | CH₃ | NHCH₃ | HCl |
| (205) | H | Cl | C₂H₅ | NHCH₃ | HCl |
| (206) | H | Cl | OCH₃ | NHCH₃ | HCl |
| (207) | H | Cl | NO₂ | NHCH₃ | HCl |
| (208) | H | Cl | NH₂ | NHCH₃ | HCl |
| (209) | H | Cl | N(CH₃)₂ | NHCH₃ | HCl |
| (210) | H | Cl | H | N(CH₃)₂ | HCl |
| (211) | H | Cl | CH₃ | N(CH₃)₂ | HCl |
| (212) | H | Cl | C₂H₅ | N(CH₃)₂ | HCl |
| (213) | H | Cl | OCH₃ | N(CH₃)₂ | HCl |
| (214) | H | Cl | NO₂ | N(CH₃)₂ | HCl |
| (215) | H | Cl | NH₂ | N(CH₃)₂ | HCl |
| (216) | H | Cl | N(CH₃)₂ | N(CH₃)₂ | HCl |
| (217) | H | Cl | H | OH | HCl |
| (218) | H | Cl | CH₃ | OH | HCl |
| (219) | H | Cl | C₂H₅ | OH | HCl |
| (220) | H | Cl | OCH₃ | OH | HCl |
| (221) | H | Cl | NO₂ | OH | HCl |
| (222) | H | Cl | NH₂ | OH | HCl |
| (223) | H | Cl | N(CH₃)₂ | OH | HCl |
| (224) | H | Cl | SO₂CH₃ | CH₃ | HCl |

### Examples 225-260

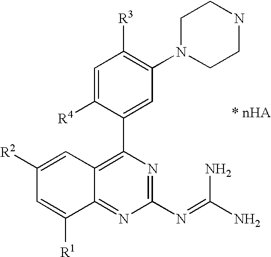

| | R¹ | R² | R³ | R⁴ | nHA | |
|---|---|---|---|---|---|---|
| (225) | H | Cl | H | H | 3 HCl | m.p. 241° C. |
| (226) | H | Cl | CH₃ | H | HCl | |
| (227) | H | Cl | C₂H₅ | H | HCl | |
| (228) | H | Cl | OCH₃ | H | HCl | |
| (229) | H | Cl | NO₂ | H | HCl | |
| (230) | H | Cl | NH₂ | H | pTsOH | |
| (231) | H | Cl | N(CH₃)₂ | H | pTsOH | |
| (232) | H | Cl | H | NH₂ | HCl | |
| (233) | H | Cl | CH₃ | NH₂ | pTsOH | |
| (234) | H | Cl | C₂H₅ | NH₂ | HCl | |
| (235) | H | Cl | OCH₃ | NH₂ | HCl | |
| (236) | H | Cl | NO₂ | NH₂ | HCl | |
| (237) | H | Cl | NH₂ | NH₂ | HCl | |
| (238) | H | Cl | N(CH₃)₂ | NH₂ | HCl | |
| (239) | H | Cl | H | NHCH₃ | HCl | |
| (240) | H | Cl | CH₃ | NHCH₃ | HCl | |
| (241) | H | Cl | C₂H₅ | NHCH₃ | HCl | |
| (242) | H | Cl | OCH₃ | NHCH₃ | HCl | |
| (243) | H | Cl | NO₂ | NHCH₃ | HCl | |
| (244) | H | Cl | NH₂ | NHCH₃ | HCl | |
| (245) | H | Cl | N(CH₃)₂ | NHCH₃ | HCl | |
| (246) | H | Cl | H | N(CH₃)₂ | HCl | |
| (247) | H | Cl | CH₃ | N(CH₃)₂ | HCl | |
| (248) | H | Cl | C₂H₅ | N(CH₃)₂ | HCl | |
| (249) | H | Cl | OCH₃ | N(CH₃)₂ | HCl | |
| (250) | H | Cl | NO₂ | N(CH₃)₂ | HCl | |
| (251) | H | Cl | NH₂ | N(CH₃)₂ | HCl | |
| (252) | H | Cl | N(CH₃)₂ | N(CH₃)₂ | HCl | |
| (253) | H | Cl | H | OH | HCl | |
| (254) | H | Cl | CH₃ | OH | HCl | |
| (255) | H | Cl | C₂H₅ | OH | HCl | |
| (256) | H | Cl | OCH₃ | OH | HCl | |
| (257) | H | Cl | NO₂ | OH | HCl | |
| (258) | H | Cl | NH₂ | OH | HCl | |
| (259) | H | Cl | N(CH₃)₂ | OH | HCl | |
| (260) | H | Cl | SO₂CH₃ | CH₃ | HCl | |

### Examples 261-296

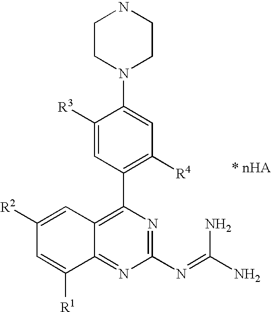

| | R¹ | R² | R³ | R⁴ | nHA |
|---|---|---|---|---|---|
| (261) | H | Cl | H | H | 2 pTsOH |
| (262) | H | Cl | CH₃ | H | HCl |
| (263) | H | Cl | C₂H₅ | H | HCl |
| (264) | H | Cl | OCH₃ | H | HCl |
| (265) | H | Cl | NO₂ | H | HCl |
| (266) | H | Cl | NH₂ | H | pTsOH |
| (267) | H | Cl | N(CH₃)₂ | H | pTsOH |
| (268) | H | Cl | H | NH₂ | HCl |
| (269) | H | Cl | CH₃ | NH₂ | pTsOH |
| (270) | H | Cl | C₂H₅ | NH₂ | HCl |
| (271) | H | Cl | OCH₃ | NH₂ | HCl |
| (272) | H | Cl | NO₂ | NH₂ | HCl |
| (273) | H | Cl | NH₂ | NH₂ | HCl |
| (274) | H | Cl | N(CH₃)₂ | NH₂ | HCl |
| (275) | H | Cl | H | NHCH₃ | HCl |
| (276) | H | Cl | CH₃ | NHCH₃ | HCl |
| (277) | H | Cl | C₂H₅ | NHCH₃ | HCl |
| (278) | H | Cl | OCH₃ | NHCH₃ | HCl |
| (279) | H | Cl | NO₂ | NHCH₃ | HCl |
| (280) | H | Cl | NH₂ | NHCH₃ | HCl |
| (281) | H | Cl | N(CH₃)₂ | NHCH₃ | HCl |
| (282) | H | Cl | H | N(CH₃)₂ | HCl |
| (283) | H | Cl | CH₃ | N(CH₃)₂ | HCl |
| (284) | H | Cl | C₂H₅ | N(CH₃)₂ | HCl |
| (285) | H | Cl | OCH₃ | N(CH₃)₂ | HCl |
| (286) | H | Cl | NO₂ | N(CH₃)₂ | HCl |
| (287) | H | Cl | NH₂ | N(CH₃)₂ | HCl |
| (288) | H | Cl | N(CH₃)₂ | N(CH₃)₂ | HCl |
| (289) | H | Cl | H | OH | HCl |
| (290) | H | Cl | CH₃ | OH | HCl |
| (291) | H | Cl | C₂H₅ | OH | HCl |
| (292) | H | Cl | OCH₃ | OH | HCl |
| (293) | H | Cl | NO₂ | OH | HCl |
| (294) | H | Cl | NH₂ | OH | HCl |
| (295) | H | Cl | N(CH₃)₂ | OH | HCl |
| (296) | H | Cl | SO₂CH₃ | CH₃ | HCl |

### Examples 297-332

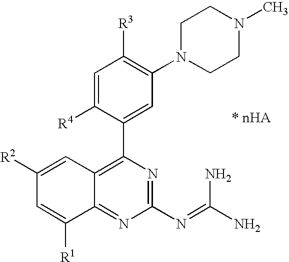

| | R¹ | R² | R³ | R⁴ | nHA | |
|---|---|---|---|---|---|---|
| (297) | H | Cl | H | H | 2 HCl | m.p. 137° C. |
| (298) | H | Cl | CH₃ | H | HCl | |
| (299) | H | Cl | C₂H₅ | H | HCl | |
| (300) | H | Cl | OCH₃ | H | HCl | |
| (301) | H | Cl | NO₂ | H | HCl | |
| (302) | H | Cl | NH₂ | H | pTsOH | |
| (303) | H | Cl | N(CH₃)₂ | H | pTsOH | |
| (304) | H | Cl | H | NH₂ | HCl | |
| (305) | H | Cl | CH₃ | NH₂ | pTsOH | |
| (306) | H | Cl | C₂H₅ | NH₂ | HCl | |
| (307) | H | Cl | OCH₃ | NH₂ | HCl | |
| (308) | H | Cl | NO₂ | NH₂ | HCl | |
| (309) | H | Cl | NH₂ | NH₂ | HCl | |
| (310) | H | Cl | N(CH₃)₂ | NH₂ | HCl | |
| (311) | H | Cl | H | NHCH₃ | HCl | |
| (312) | H | Cl | CH₃ | NHCH₃ | HCl | |
| (313) | H | Cl | C₂H₅ | NHCH₃ | HCl | |
| (314) | H | Cl | OCH₃ | NHCH₃ | HCl | |
| (315) | H | Cl | NO₂ | NHCH₃ | HCl | |
| (316) | H | Cl | NH₂ | NHCH₃ | HCl | |
| (317) | H | Cl | N(CH₃)₂ | NHCH₃ | HCl | |
| (318) | H | Cl | H | N(CH₃)₂ | HCl | |
| (319) | H | Cl | CH₃ | N(CH₃)₂ | HCl | |
| (320) | H | Cl | C₂H₅ | N(CH₃)₂ | HCl | |
| (321) | H | Cl | OCH₃ | N(CH₃)₂ | HCl | |
| (322) | H | Cl | NO₂ | N(CH₃)₂ | HCl | |
| (323) | H | Cl | NH₂ | N(CH₃)₂ | HCl | |
| (324) | H | Cl | N(CH₃)₂ | N(CH₃)₂ | HCl | |
| (325) | H | Cl | H | OH | HCl | |
| (326) | H | Cl | CH₃ | OH | HCl | |
| (327) | H | Cl | C₂H₅ | OH | HCl | |
| (328) | H | Cl | OCH₃ | OH | HCl | |
| (329) | H | Cl | NO₂ | OH | HCl | |
| (330) | H | Cl | NH₂ | OH | HCl | |
| (331) | H | Cl | N(CH₃)₂ | OH | HCl | |
| (332) | H | Cl | SO₂CH₃ | CH₃ | HCl | |

### Examples 333-368

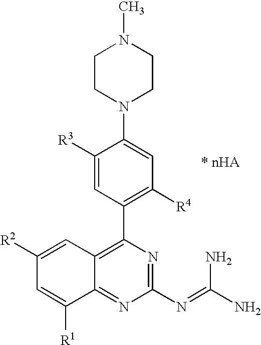

| | R¹ | R² | R³ | R⁴ | nHA | |
|---|---|---|---|---|---|---|
| (333) | H | Cl | H | H | 3 HCl | m.p. >160° C. |
| (334) | H | Cl | CH₃ | H | HCl | |
| (335) | H | Cl | C₂H₅ | H | HCl | |
| (336) | H | Cl | OCH₃ | H | HCl | |
| (337) | H | Cl | NO₂ | H | HCl | |
| (338) | H | Cl | NH₂ | H | pTsOH | |
| (339) | H | Cl | N(CH₃)₂ | H | pTsOH | |
| (340) | H | Cl | H | NH₂ | HCl | |
| (341) | H | Cl | CH₃ | NH₂ | pTsOH | |
| (342) | H | Cl | C₂H₅ | NH₂ | HCl | |
| (343) | H | Cl | OCH₃ | NH₂ | HCl | |
| (344) | H | Cl | NO₂ | NH₂ | HCl | |
| (345) | H | Cl | NH₂ | NH₂ | HCl | |
| (346) | H | Cl | N(CH₃)₂ | NH₂ | HCl | |
| (347) | H | Cl | H | NHCH₃ | HCl | |
| (348) | H | Cl | CH₃ | NHCH₃ | HCl | |
| (349) | H | Cl | C₂H₅ | NHCH₃ | HCl | |
| (350) | H | Cl | OCH₃ | NHCH₃ | HCl | |
| (351) | H | Cl | NO₂ | NHCH₃ | HCl | |
| (352) | H | Cl | NH₂ | NHCH₃ | HCl | |
| (353) | H | Cl | N(CH₃)₂ | NHCH₃ | HCl | |
| (354) | H | Cl | H | N(CH₃)₂ | HCl | |
| (355) | H | Cl | CH₃ | N(CH₃)₂ | HCl | |
| (356) | H | Cl | C₂H₅ | N(CH₃)₂ | HCl | |
| (357) | H | Cl | OCH₃ | N(CH₃)₂ | HCl | |
| (358) | H | Cl | NO₂ | N(CH₃)₂ | HCl | |
| (359) | H | Cl | NH₂ | N(CH₃)₂ | HCl | |
| (360) | H | Cl | N(CH₃)₂ | N(CH₃)₂ | HCl | |
| (361) | H | Cl | H | OH | HCl | |
| (362) | H | Cl | CH₃ | OH | HCl | |
| (363) | H | Cl | C₂H₅ | OH | HCl | |
| (364) | H | Cl | OCH₃ | OH | HCl | |
| (365) | H | Cl | NO₂ | OH | HCl | |
| (366) | H | Cl | NH₂ | OH | HCl | |
| (367) | H | Cl | N(CH₃)₂ | OH | HCl | |
| (368) | H | Cl | SO₂CH₃ | CH₃ | HCl | |

### Examples 369-404

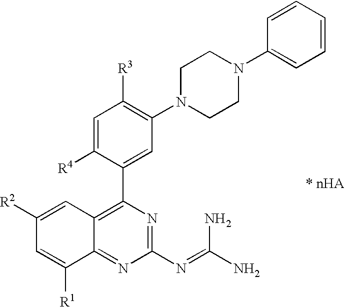

| | R¹ | R² | R³ | R⁴ | nHA |
|---|---|---|---|---|---|
| (369) | H | Cl | H | H | pTsOH |
| (370) | H | Cl | CH₃ | H | HCl |
| (371) | H | Cl | C₂H₅ | H | HCl |
| (372) | H | Cl | OCH₃ | H | HCl |
| (373) | H | Cl | NO₂ | H | HCl |
| (374) | H | Cl | NH₂ | H | pTsOH |
| (375) | H | Cl | N(CH₃)₂ | H | pTsOH |
| (376) | H | Cl | H | NH₂ | HCl |
| (377) | H | Cl | CH₃ | NH₂ | pTsOH |
| (378) | H | Cl | C₂H₅ | NH₂ | HCl |
| (379) | H | Cl | OCH₃ | NH₂ | HCl |
| (380) | H | Cl | NO₂ | NH₂ | HCl |
| (381) | H | Cl | NH₂ | NH₂ | HCl |
| (382) | H | Cl | N(CH₃)₂ | NH₂ | HCl |
| (383) | H | Cl | H | NHCH₃ | HCl |
| (384) | H | Cl | CH₃ | NHCH₃ | HCl |
| (385) | H | Cl | C₂H₅ | NHCH₃ | HCl |
| (386) | H | Cl | OCH₃ | NHCH₃ | HCl |
| (387) | H | Cl | NO₂ | NHCH₃ | HCl |
| (388) | H | Cl | NH₂ | NHCH₃ | HCl |
| (389) | H | Cl | N(CH₃)₂ | NHCH₃ | HCl |
| (390) | H | Cl | H | N(CH₃)₂ | HCl |
| (391) | H | Cl | CH₃ | N(CH₃)₂ | HCl |
| (392) | H | Cl | C₂H₅ | N(CH₃)₂ | HCl |
| (393) | H | Cl | OCH₃ | N(CH₃)₂ | HCl |
| (394) | H | Cl | NO₂ | N(CH₃)₂ | HCl |
| (395) | H | Cl | NH₂ | N(CH₃)₂ | HCl |
| (396) | H | Cl | N(CH₃)₂ | N(CH₃)₂ | HCl |
| (397) | H | Cl | H | OH | HCl |
| (398) | H | Cl | CH₃ | OH | HCl |
| (399) | H | Cl | C₂H₅ | OH | HCl |
| (400) | H | Cl | OCH₃ | OH | HCl |
| (401) | H | Cl | NO₂ | OH | HCl |
| (402) | H | Cl | NH₂ | OH | HCl |
| (403) | H | Cl | N(CH₃)₂ | OH | HCl |
| (404) | H | Cl | SO₂CH₃ | CH₃ | HCl |

### Examples 405-440

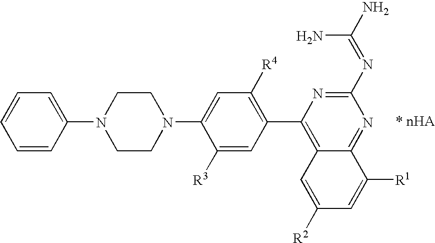

| | R¹ | R² | R³ | R⁴ | nHA | |
|---|---|---|---|---|---|---|
| (405) | H | Cl | H | H | 3 HCl | m.p. 240° C. |
| (406) | H | Cl | CH₃ | H | HCl | |
| (407) | H | Cl | C₂H₅ | H | HCl | |
| (408) | H | Cl | OCH₃ | H | HCl | |
| (409) | H | Cl | NO₂ | H | HCl | |
| (410) | H | Cl | NH₂ | H | pTsOH | |
| (411) | H | Cl | N(CH₃)₂ | H | pTsOH | |
| (412) | H | Cl | H | NH₂ | HCl | |
| (413) | H | Cl | CH₃ | NH₂ | pTsOH | |
| (414) | H | Cl | C₂H₅ | NH₂ | HCl | |
| (415) | H | Cl | OCH₃ | NH₂ | HCl | |
| (416) | H | Cl | NO₂ | NH₂ | HCl | |
| (417) | H | Cl | NH₂ | NH₂ | HCl | |
| (418) | H | Cl | N(CH₃)₂ | NH₂ | HCl | |
| (419) | H | Cl | H | NHCH₃ | HCl | |
| (420) | H | Cl | CH₃ | NHCH₃ | HCl | |
| (421) | H | Cl | C₂H₅ | NHCH₃ | HCl | |
| (422) | H | Cl | OCH₃ | NHCH₃ | HCl | |
| (423) | H | Cl | NO₂ | NHCH₃ | HCl | |
| (424) | H | Cl | NH₂ | NHCH₃ | HCl | |
| (425) | H | Cl | N(CH₃)₂ | NHCH₃ | HCl | |
| (426) | H | Cl | H | N(CH₃)₂ | HCl | |
| (427) | H | Cl | CH₃ | N(CH₃)₂ | HCl | |
| (428) | H | Cl | C₂H₅ | N(CH₃)₂ | HCl | |
| (429) | H | Cl | OCH₃ | N(CH₃)₂ | HCl | |
| (430) | H | Cl | NO₂ | N(CH₃)₂ | HCl | |
| (431) | H | Cl | NH₂ | N(CH₃)₂ | HCl | |
| (432) | H | Cl | N(CH₃)₂ | N(CH₃)₂ | HCl | |
| (433) | H | Cl | H | OH | HCl | |
| (434) | H | Cl | CH₃ | OH | HCl | |
| (435) | H | Cl | C₂H₅ | OH | HCl | |
| (436) | H | Cl | OCH₃ | OH | HCl | |
| (437) | H | Cl | NO₂ | OH | HCl | |
| (438) | H | Cl | NH₂ | OH | HCl | |
| (439) | H | Cl | N(CH₃)₂ | OH | HCl | |
| (440) | H | Cl | SO₂CH₃ | CH₃ | HCl | |

### Examples 441-476

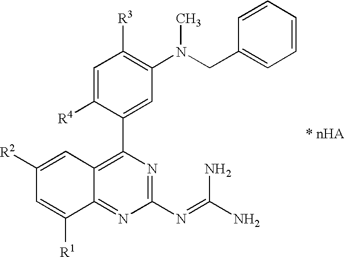

| | R¹ | R² | R³ | R⁴ | nHA |
|---|---|---|---|---|---|
| (441) | H | Cl | H | H | pTsOH |
| (442) | H | Cl | CH₃ | H | HCl |
| (443) | H | Cl | C₂H₅ | H | HCl |
| (444) | H | Cl | OCH₃ | H | HCl |
| (445) | H | Cl | NO₂ | H | HCl |
| (446) | H | Cl | NH₂ | H | pTsOH |
| (447) | H | Cl | N(CH₃)₂ | H | pTsOH |
| (448) | H | Cl | H | NH₂ | HCl |
| (449) | H | Cl | CH₃ | NH₂ | pTsOH |
| (450) | H | Cl | C₂H₅ | NH₂ | HCl |
| (451) | H | Cl | OCH₃ | NH₂ | HCl |
| (452) | H | Cl | NO₂ | NH₂ | HCl |
| (453) | H | Cl | NH₂ | NH₂ | HCl |
| (454) | H | Cl | N(CH₃)₂ | NH₂ | HCl |
| (455) | H | Cl | H | NHCH₃ | HCl |
| (456) | H | Cl | CH₃ | NHCH₃ | HCl |
| (457) | H | Cl | C₂H₅ | NHCH₃ | HCl |
| (458) | H | Cl | OCH₃ | NHCH₃ | HCl |
| (459) | H | Cl | NO₂ | NHCH₃ | HCl |
| (460) | H | Cl | NH₂ | NHCH₃ | HCl |
| (461) | H | Cl | N(CH₃)₂ | NHCH₃ | HCl |
| (462) | H | Cl | H | N(CH₃)₂ | HCl |
| (463) | H | Cl | CH₃ | N(CH₃)₂ | HCl |
| (464) | H | Cl | C₂H₅ | N(CH₃)₂ | HCl |
| (465) | H | Cl | OCH₃ | N(CH₃)₂ | HCl |
| (466) | H | Cl | NO₂ | N(CH₃)₂ | HCl |
| (467) | H | Cl | NH₂ | N(CH₃)₂ | HCl |
| (468) | H | Cl | N(CH₃)₂ | N(CH₃)₂ | HCl |
| (469) | H | Cl | H | OH | HCl |
| (470) | H | Cl | CH₃ | OH | HCl |
| (471) | H | Cl | C₂H₅ | OH | HCl |
| (472) | H | Cl | OCH₃ | OH | HCl |
| (473) | H | Cl | NO₂ | OH | HCl |
| (474) | H | Cl | NH₂ | OH | HCl |
| (475) | H | Cl | N(CH₃)₂ | OH | HCl |
| (476) | H | Cl | SO₂CH₃ | CH₃ | HCl |

### Examples 477-512

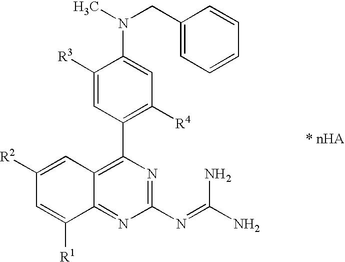

| | R¹ | R² | R³ | R⁴ | nHA | |
|---|---|---|---|---|---|---|
| (477) | H | Cl | H | H | HCl | m.p. 170° C. |
| (478) | H | Cl | CH₃ | H | HCl | |
| (479) | H | Cl | C₂H₅ | H | HCl | |
| (480) | H | Cl | OCH₃ | H | HCl | |
| (481) | H | Cl | NO₂ | H | HCl | |
| (482) | H | Cl | NH₂ | H | pTsOH | |
| (483) | H | Cl | N(CH₃)₂ | H | pTsOH | |
| (484) | H | Cl | H | NH₂ | HCl | |
| (485) | H | Cl | CH₃ | NH₂ | pTsOH | |
| (486) | H | Cl | C₂H₅ | NH₂ | HCl | |
| (487) | H | Cl | OCH₃ | NH₂ | HCl | |
| (488) | H | Cl | NO₂ | NH₂ | HCl | |
| (489) | H | Cl | NH₂ | NH₂ | HCl | |
| (490) | H | Cl | N(CH₃)₂ | NH₂ | HCl | |
| (491) | H | Cl | H | NHCH₃ | HCl | |
| (492) | H | Cl | CH₃ | NHCH₃ | HCl | |
| (493) | H | Cl | C₂H₅ | NHCH₃ | HCl | |
| (494) | H | Cl | OCH₃ | NHCH₃ | HCl | |
| (495) | H | Cl | NO₂ | NHCH₃ | HCl | |
| (496) | H | Cl | NH₂ | NHCH₃ | HCl | |
| (497) | H | Cl | N(CH₃)₂ | NHCH₃ | HCl | |
| (498) | H | Cl | H | N(CH₃)₂ | HCl | |
| (499) | H | Cl | CH₃ | N(CH₃)₂ | HCl | |
| (500) | H | Cl | C₂H₅ | N(CH₃)₂ | HCl | |
| (501) | H | Cl | OCH₃ | N(CH₃)₂ | HCl | |
| (502) | H | Cl | NO₂ | N(CH₃)₂ | HCl | |
| (503) | H | Cl | NH₂ | N(CH₃)₂ | HCl | |
| (504) | H | Cl | N(CH₃)₂ | N(CH₃)₂ | HCl | |
| (505) | H | Cl | H | OH | HCl | |
| (506) | H | Cl | CH₃ | OH | HCl | |
| (507) | H | Cl | C₂H₅ | OH | HCl | |
| (508) | H | Cl | OCH₃ | OH | HCl | |
| (509) | H | Cl | NO₂ | OH | HCl | |
| (510) | H | Cl | NH₂ | OH | HCl | |
| (511) | H | Cl | N(CH₃)₂ | OH | HCl | |
| (512) | H | Cl | SO₂CH₃ | CH₃ | HCl | |

### Examples 513-548

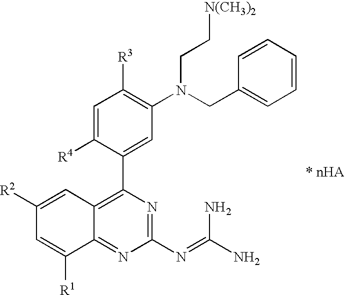

| | R¹ | R² | R³ | R⁴ | nHA | |
|---|---|---|---|---|---|---|
| (513) | H | Cl | H | H | 2 HCl | m.p. 207° C. |
| (514) | H | Cl | CH₃ | H | HCl | |
| (515) | H | Cl | C₂H₅ | H | HCl | |
| (516) | H | Cl | OCH₃ | H | HCl | |
| (517) | H | Cl | NO₂ | H | HCl | |
| (518) | H | Cl | NH₂ | H | pTsOH | |
| (519) | H | Cl | N(CH₃)₂ | H | pTsOH | |
| (520) | H | Cl | H | NH₂ | HCl | |
| (521) | H | Cl | CH₃ | NH₂ | pTsOH | |
| (522) | H | Cl | C₂H₅ | NH₂ | HCl | |
| (523) | H | Cl | OCH₃ | NH₂ | HCl | |
| (524) | H | Cl | NO₂ | NH₂ | HCl | |
| (525) | H | Cl | NH₂ | NH₂ | HCl | |
| (526) | H | Cl | N(CH₃)₂ | NH₂ | HCl | |
| (527) | H | Cl | H | NHCH₃ | HCl | |
| (528) | H | Cl | CH₃ | NHCH₃ | HCl | |
| (529) | H | Cl | C₂H₅ | NHCH₃ | HCl | |
| (530) | H | Cl | OCH₃ | NHCH₃ | HCl | |
| (531) | H | Cl | NO₂ | NHCH₃ | HCl | |
| (532) | H | Cl | NH₂ | NHCH₃ | HCl | |
| (533) | H | Cl | N(CH₃)₂ | NHCH₃ | HCl | |
| (534) | H | Cl | H | N(CH₃)₂ | HCl | |
| (535) | H | Cl | CH₃ | N(CH₃)₂ | HCl | |
| (536) | H | Cl | C₂H₅ | N(CH₃)₂ | HCl | |
| (537) | H | Cl | OCH₃ | N(CH₃)₂ | HCl | |
| (538) | H | Cl | NO₂ | N(CH₃)₂ | HCl | |
| (539) | H | Cl | NH₂ | N(CH₃)₂ | HCl | |
| (540) | H | Cl | N(CH₃)₂ | N(CH₃)₂ | HCl | |
| (541) | H | Cl | H | OH | HCl | |
| (542) | H | Cl | CH₃ | OH | HCl | |
| (543) | H | Cl | C₂H₅ | OH | HCl | |
| (544) | H | Cl | OCH₃ | OH | HCl | |
| (545) | H | Cl | NO₂ | OH | HCl | |
| (546) | H | Cl | NH₂ | OH | HCl | |
| (547) | H | Cl | N(CH₃)₂ | OH | HCl | |
| (548) | H | Cl | SO₂CH₃ | CH₃ | HCl | |

### Examples 549-584

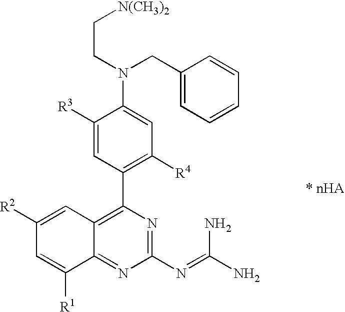

| | R¹ | R² | R³ | R⁴ | nHA |
|---|---|---|---|---|---|
| (549) | H | Cl | H | H | pTsQH |
| (550) | H | Cl | CH₃ | H | HCl |
| (551) | H | Cl | C₂H₅ | H | HCl |
| (552) | H | Cl | OCH₃ | H | HCl |
| (553) | H | Cl | NO₂ | H | HCl |
| (554) | H | Cl | NH₂ | H | pTsOH |
| (555) | H | Cl | N(CH₃)₂ | H | pTsOH |
| (556) | H | Cl | H | NH₂ | HCl |
| (557) | H | Cl | CH₃ | NH₂ | pTsOH |
| (558) | H | Cl | C₂H₅ | NH₂ | HCl |
| (559) | H | Cl | OCH₃ | NH₂ | HCl |
| (560) | H | Cl | NO₂ | NH₂ | HCl |
| (561) | H | Cl | NH₂ | NH₂ | HCl |
| (562) | H | Cl | N(CH₃)₂ | NH₂ | HCl |
| (563) | H | Cl | H | NHCH₃ | HCl |
| (564) | H | Cl | CH₃ | NHCH₃ | HCl |
| (565) | H | Cl | C₂H₅ | NHCH₃ | HCl |
| (566) | H | Cl | OCH₃ | NHCH₃ | HCl |
| (567) | H | Cl | NO₂ | NHCH₃ | HCl |
| (568) | H | Cl | NH₂ | NHCH₃ | HCl |
| (569) | H | Cl | N(CH₃)₂ | NHCH₃ | HCl |
| (570) | H | Cl | H | N(CH₃)₂ | HCl |
| (571) | H | Cl | CH₃ | N(CH₃)₂ | HCl |
| (572) | H | Cl | C₂H₅ | N(CH₃)₂ | HCl |
| (573) | H | Cl | OCH₃ | N(CH₃)₂ | HCl |
| (574) | H | Cl | NO₂ | N(CH₃)₂ | HCl |
| (575) | H | Cl | NH₂ | N(CH₃)₂ | HCl |
| (576) | H | Cl | N(CH₃)₂ | N(CH₃)₂ | HCl |
| (577) | H | Cl | H | OH | HCl |
| (578) | H | Cl | CH₃ | OH | HCl |
| (579) | H | Cl | C₂2H₅ | OH | HCl |
| (580) | H | Cl | OCH₃ | OH | HCl |
| (581) | H | Cl | NO₂ | OH | HCl |
| (582) | H | Cl | NH₂ | OH | HCl |
| (583) | H | Cl | N(CH₃)₂ | OH | HCl |
| (584) | H | Cl | SO₂CH₃ | OH₃ | HCl |

Pharmacological Tests

The method used for the characterisation of the compounds of the formula I as NHE-3 inhibitors is described below.

The compounds of the formula I are characterised with respect to their selectivity for the NHE-1 to NHE-3 isoforms. The three isoforms are expressed in stable form in mouse fibroblast cell lines. The inhibitory action of the compounds is assessed by determination of the EIPA-sensitive uptake of ²²Na⁺ into the cells after intracellular acidosis.

Material and Methods

LAP1 Cell Lines Which Express the Different NHE Isoforms

The LAP1 cell lines which express the NHE-1, -2 and -3 isoforms (a mouse fibroblast cell line) is obtained from Prof. J. Pouysségur (Nice, France). The transfection is carried out by the method of Franchi et al. (1986). The cells are cultivated in Dulbeccos modified eagle medium (DMEM) with 10% of deactivated foetal calf serum (FCS). For selection of the NHE-expressing cells, the so-called “acid killing method” of Sardet et al. (1989) is used. The cells are firstly incubated for 30 minutes in an NH₄Cl-containing bicarbonate- and sodium-free buffer. The extracellular NH₄Cl is then removed by washing with a bicarbonate-, NH₄Cl- and sodium-free buffer. The cells are subsequently incubated in a bicarbonate-free NaCl-containing buffer. Only those cells which functionally express NHE are able to survive the intracellular acidification to which they are subjected.

Characterisation of NHE Inhibitors with Respect to Their Isoform Selectivity

With the above-mentioned mouse fibroblast cell lines which express the NHE-1, NHE-2 and NHE-3 isoforms, compounds are tested for selectivity with respect to the isoforms by the procedure described by Counillon et al. (1993) and Scholz et al. (1995). The cells are acidified intracellularly by the NH₄Cl prepulse method and subsequently by incubation in a bicarbonate-free ²²Na⁺-containing buffer. Owing to the intracellular acidification, NHE is activated, and sodium is taken up into the cells. The effect of the test compound is expressed as inhibition of EIPA (ethylisopropylamiloride)-sensitive ²²Na⁺ take-up.

The cells which expressed NHE-1, NHE-2 and NHE-3 are sown out in a density of 5-7.5×10⁴ cells/well in 24-well microtitre plates and cultured to confluence for from 24 to 48 hours. The medium is removed by suction, and the cells are incubated for 60 minutes at 37° C. in NH₄Cl buffer (50 mM NH₄Cl, 70 mM choline chloride, 15 mM MOPS, pH 7.0). The buffer is subsequently removed, and the cells are rapidly covered twice with the choline chloride wash buffer (120 mM choline chloride, 15 mM PIPES/tris, 0.1 mM ouabain, 1 mM MgCl₂, 2 mM CaCl₂, pH 7.4) and filtered off with suction. The cells are subsequently covered with the choline chloride charging buffer (120 mM choline chloride, 15 mM PIPES/tris, 0.1 mM PIPES/tris, 0.1 mM ouabain, 1 mM MgCl₂, 2 mM CaCl₂, pH 7.4, ²²Na⁺ (0.925 kBg/100 ml of charging buffer)) and then incubated in this buffer for 6 minutes. After expiry of the incubation time, the incubation buffer is removed by suction. In order to remove extracellular radioactivity, the cells are washed rapidly four times with ice-cold phosphate-buffered saline solution (PBS). The cells are then solubilised by addition of 0.3 ml of 0.1 N NaOH per well. The cell fragment-containing solutions are transferred into scintillation tubes. Each well is then washed twice with 0.3 ml of 0.1 N NaOH, and the washing solutions are likewise introduced into the corresponding scintillation tubes. Scintillation cocktail is added to the tubes containing the cell lysate, and the radioactivity taken up into the cells is determined by determination of the p radiation.

Literature:

Counillon et al. (1993) Mol. Pharmacol. 44: 1041-1045
Franchi et al. (1986) Proc. Natl. Acad. Sci. USA 83: 9388-9392
Sardet et al. (1989) Cell 56: 271-280
Scholz et al. (1995) Cardiovasc. Res. 29: 260-268

The examples below relate to pharmaceutical preparations:

### Example A

### Injection Vials

A solution of 100 g of an NHE-3 inhibitor of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions.

Each injection vial contains 5 mg of active ingredient.

### Example B

### Suppositories

A mixture of 20 g of an NHE-3 inhibitor of the formula I is melted with 100 g of soya lecithin and 1400 g of cocoa butter, poured into moulds and allowed to cool. Each suppository contains 20 mg of active ingredient.

### Example C

### Solution

A solution is prepared from 1 g of an NHE-3 inhibitor of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D

### Ointment

500 mg of an NHE-3 inhibitor of the formula I are mixed with 99.5 g of Vaseline under aseptic conditions.

### Example E

### Tablets

A mixture of 1 kg of an NHE-3 inhibitor of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, tragacanth and dye.

### Example G

### Capsules

2 kg of an NHE-3 inhibitor of the formula I are introduced into hard gelatine capsules in a conventional manner in such a way that each capsule contains 20 mg of the active ingredient.

### Example H

### Ampoules

A solution of 1 kg of an NHE-3 inhibitor of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 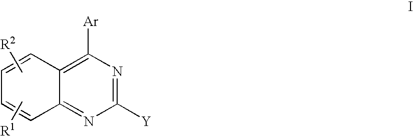
in which
Ar is X-substituted phenyl or naphthyl, which is additionally substituted by R³ and R⁴,
Y is 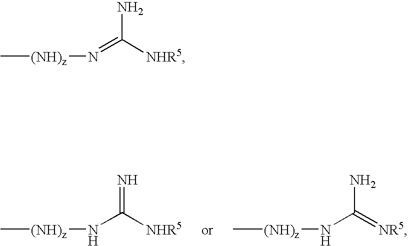
X is H, NR⁶R⁷ or a saturated 5-7-membered ring having two N atoms,
R¹, R², R³
and R⁴ are each, independently of one another, H, A, OA, Hal, CF₃, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃,
A is alkyl having 1, 2, 3, 4, 5 or 6 carbon atoms,
Hal is F, Cl, Br or I,
R⁵ is H, A, OH, NO₂, phenyl which is unsubstituted or monosubstituted or polysubstituted by A, OA, Hal or CF₃, an amino-protecting group or 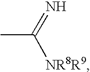
R⁶ and R⁷ are each, independently of one another, H, A, phenyl which is unsubstituted or monosubstituted or polysubstituted by A, OA, Hal or CF₃, benzyl, an amino-protecting group or —(CH₂)ₙNR¹⁰R¹¹,
R⁸ and R⁹ are each, independently of one another, H or A,
R¹⁰
and R¹¹ are each, independently of one another, H, A, phenyl which is unsubstituted or monosubstituted or polysubstituted by A, OA, Hal or CF₃, benzyl or an amino-protecting group,
z is 0 or 1, and
n is 2, 3 or 4,
and salts, solvates and stereoisomers thereof, including mixtures thereof in all ratios, and pharmaceutically usable derivatives thereof, in particular physiologically tolerated salts and solvates thereof,
with the proviso that compounds of the formula I in which X is H and simultaneously z is 0 are excluded.

2. Compounds of the formula I according to claim 1, characterised in that Ar has one of the following meanings: 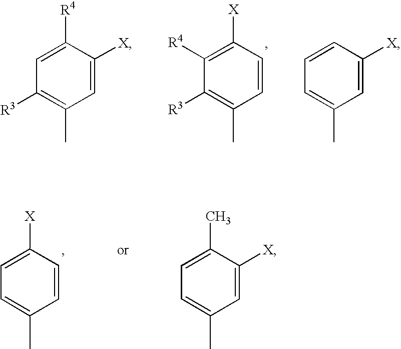
in which R³, R⁴ and X are as defined above.

3. Compounds of the formula I according to claim 1, characterised in that at least one of the radicals R¹, R², R³ and R⁴ has one of the following meanings:
Hal, A, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃.

4. Compounds of the formula I according to claim 1, characterised in that R⁵ is H, A, OH, NO₂ or an amino-protecting group.

5. Compounds of the formula I according to claim 1, characterised in that R⁶ and R⁷ are simultaneously H, independently of one another H, A, benzyl or —(CH₂)ₙNA₂.

6. Compounds of the formula I according to claim 1, characterised in that R⁸ and R⁹ are H or methyl.

7. Compounds of the formula I according to claim 1, characterised in that R¹⁰ and R¹¹ are H, A, benzyl or phenyl.

8. Compounds of the formula I according to claim 1, characterised in that X is NR⁶R⁷, a 5- to 7-membered ring having 2 N atoms or the following radical: 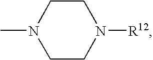
in which R¹² is H, A, Ph, benzyl or an amino-protecting group.

9. Compounds of the formulae IA, IB and IC: 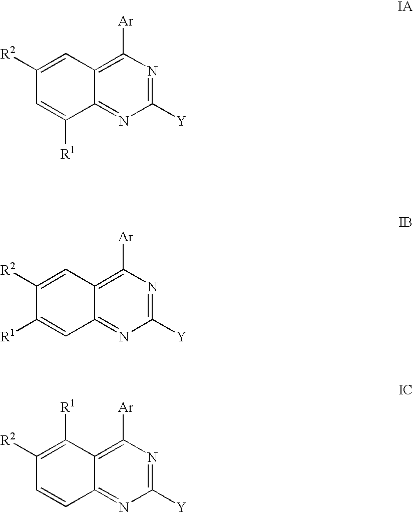
in which R¹, R², Het and Y are as defined in claim 1.

10. Compounds of the formulae IA, IB and IC according to claim 9, in which R² is Cl.

11. Compounds of the formulae I1 to I10 and salts and solvates thereof:

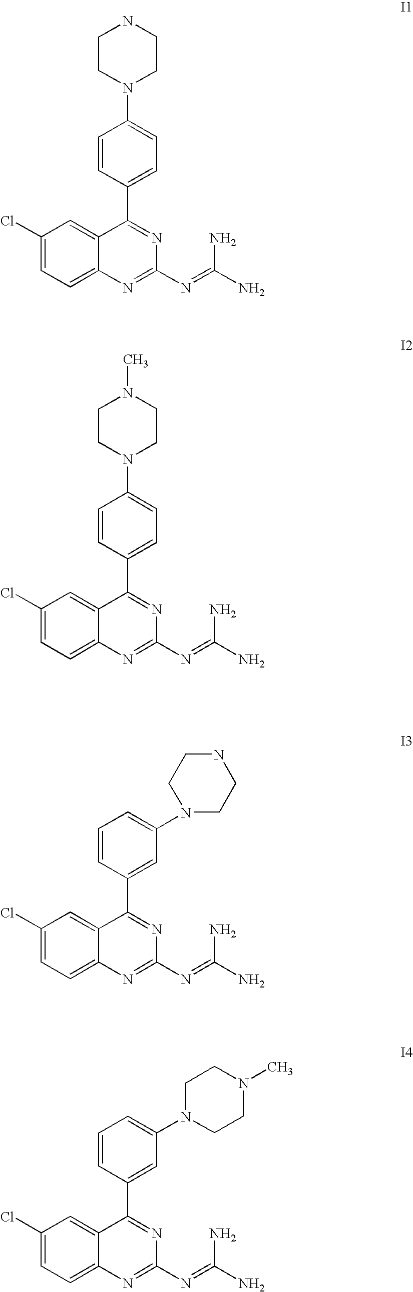

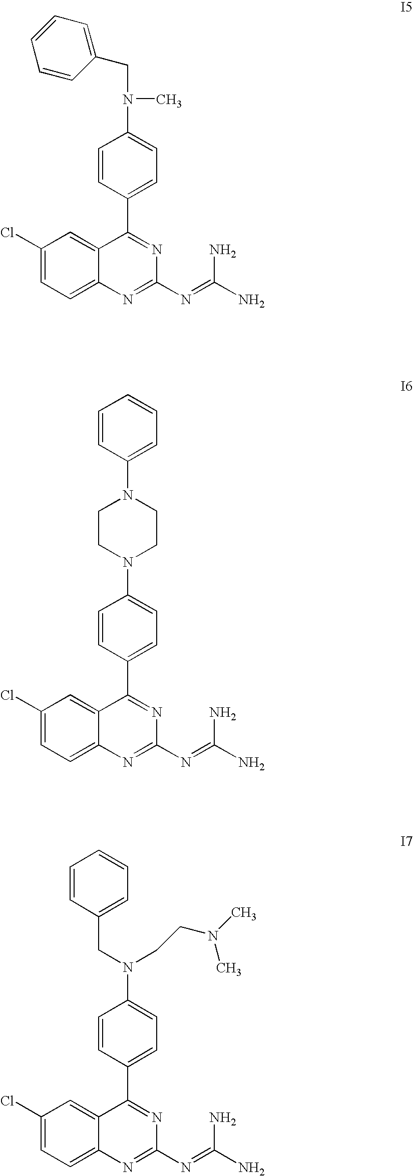

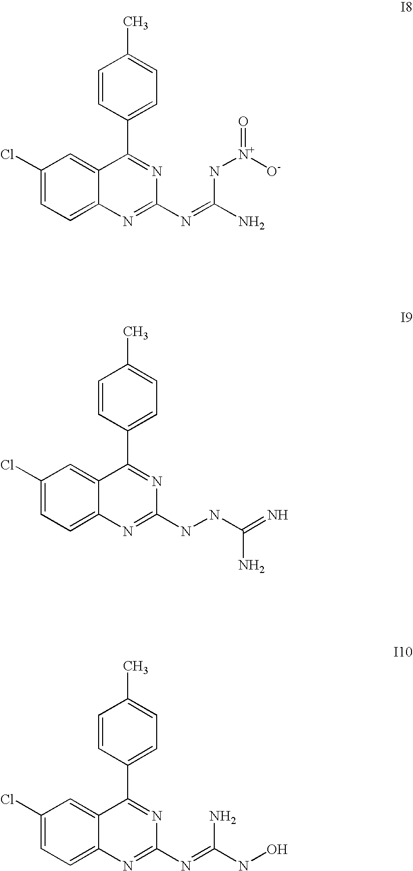

12. Compounds of the formula I according to one or more of the preceding claims and their salts and/or solvates as NHE-3 inhibitors.

13. Compounds of the formula I according to claim 1 and their physiologically acceptable salts and/or solvates for use in combating illnesses.

14. Use of compounds of the formula I according to claim 1 and/or their physiologically acceptable salts or solvates for the preparation of a medicament.

15. Use of compounds of the formula I according to claim 1 and/or their physiologically acceptable salts and/or solvates for the preparation of a medicament for the treatment and prophylaxis of hypertonia, thrombosis, ischaemic states of the heart, of the peripheral and central nervous system and of strokes, ischaemic states of peripheral organs and limbs, and for the treatment of shock states.

16. Use of compounds of the formula I according to claim 1 and/or their physiologically acceptable salts and/or solvates for the preparation of a medicament for use in surgical operations and organ transplants and for the preservation and storage of transplants for surgical measures.

17. Use of compounds of the formula I according to claim 1 and/or their physiologically acceptable salts and/or solvates for the preparation of a medicament for the treatment and prophylaxis of illnesses in which cell proliferation represents a primary or secondary cause, for the treatment or prophylaxis of disorders of fat metabolism or disturbed breathing drive.

18. Use of compounds of the formula I according to claim 1 and/or and their physiologically acceptable salts and/or solvates for the preparation of a medicament for the treatment and prophylaxis of renal ischaemia, ischaemic intestinal illnesses or for the prophylaxis of acute or chronic renal illnesses.

19. Use of compounds of the formula I according to claim 1 and/or their physiologically acceptable salts and/or solvates for the preparation of a medicament for the treatment and prophylaxis of bacterial and parasitic illnesses.

20. Pharmaceutical preparation, characterised by a content of at least one NHE-3 inhibitor according to claim 1 and/or one of its physiologically acceptable salts and/or solvates.

21. Process for the preparation of pharmaceutical preparations, characterised in that at least one compound of the formula I according to claim 1 and/or one of its physiologically acceptable salts and solvates is converted into a suitable dosage form together with at least one solid, liquid or semi-liquid excipient or adjuvant.

22. Use of compounds of the formula I according to claim 1 and/or physiologically acceptable salts and/or solvates thereof for the preparation of a medicament for the treatment and prophylaxis of diseases which are caused by increased NHE activity and/or can be influenced by a reduction in NHE activity.

23. Use of compounds of the formula I according to claim 1 and/or physiologically acceptable salts and/or solvates thereof for the preparation of a medicament for the treatment and prophylaxis of diseases or states which are caused by increased uptake of sodium ions and water in cells by organs which are undersupplied with oxygen.

24. Medicament comprising at least one compound of the formula I according to claim 1 and/or physiologically acceptable salts and solvates thereof and at least one further medicament active ingredient.

25. Set (kit) consisting of separate packs of
(a) an effective amount of a compound of the formula I according to claim 1 and/or physiologically acceptable salts and solvates thereof and
(b) an effective amount of a further medicament active ingredient.

26. Compounds according to claim 1 as medicament active ingredients.

27. Process for the preparation of the compounds of the formula I and salts and solvates thereof, characterised in that either
(a)
compounds of the formula II 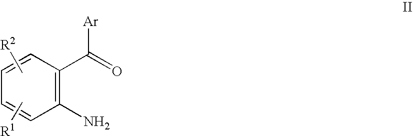
in which R¹, R² and Ar are as defined in claim 1,
are reacted with 1-cyanoguanidine or a correspondingly N-alkylated or N-arylated 1-cyanoguanidine of the formula NC—Y, in which Y is as defined in claim 1 and z is 0, or
(b)
instead of a compound of the formula NC—Y, a compound of the formula III
HN═CX—Y III
in which X is —S-alkyl, —S-aryl, —O-alkyl or —O-aryl, and Y is as defined in claim 1, where z is 0, is reacted with a compound of the formula II, or
(c)
compounds of the formula IV 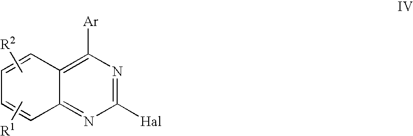
in which Ar, Hal, R¹ and R² are as defined above,
are reacted with a compound of the formula HY, in which Y is as defined in claim 1, or
(d)
compounds of the formula VI 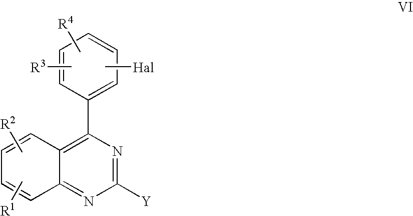
are amidated using the corresponding nitrogen bases with palladium catalysis,
and if desired, after steps (a), (b), (c) or (d), a basic or acidic compound of the formula I is converted into one of its salts or solvates by treatment with an acid or base.
